(19) United States
(12) Patent Application Publication
(10) Pub. No.: US 2011/0016548 A1
(43) Pub. Date: Jan. 20, 2011

(54) CONTROL OF ENDOGENOUS DNMT1 GENE EXPRESSION BY EXOGENOUS BINARY REGULATORY SYSTEMS

(75) Inventors: PETER W. LAIRD, SOUTH PASADENA, CA (US); KWANGHO LEE, LOS ANGELES, CA (US)

Correspondence Address: DAVIS WRIGHT TREMAINE, LLP/Seattle, 1201 Third Avenue, Suite 2200, SEATTLE, WA, 98101-3045, US

(73) Assignee: UNIVERSITY OF SOUTHERN CALIFORNIA, LOS ANGELES, CA (US)

(21) Appl. No.: 12/838,300

(22) Filed: Jul. 16, 2010

Related U.S. Application Data
(60) Provisional application No. 61/226,234, filed on Jul. 16, 2009.

(51) Int. Cl. C12N 15/87; C12N 15/63; C12N 5/10; C07K 2/00; C07K 14/00; C07K 14/035; C07H 21/04

(52) U.S. Cl. 800/22; 435/465; 435/325; 530/300; 530/324; 530/350; 536/24.1

(57) ABSTRACT

Provided are methods for controlling endogenous gene expression comprising control of the DNA methyltransferase (Dnmt1) for modulation of DNA methylation and epigenetic mechanisms. Provided are transcriptional regulatory systems involving multiple (e.g., three) exogenous binary systems, lacI, tetR and Gal4, for reversible up/down regulation of endogenous target genes Provided are lac operator and repressor modifications for improved repression relative to wild type (WT) lac and tet systems. Provided are endogenous Dnmt1 promoter modifications, comprising targeted lac operator sequences that do not significantly alter promoter activity absent repressors, yet show substantially reduced expression of the targeted allele upon lac repressor introduction. The lacO targeted Dnmt1 allele is introducible into the mouse germline, to provide a respective upregulatable transcriptional control system in vivo (e.g., two binary systems, tet operator/tetVP16 and Gal4 binding sequence/Gal4VP16, and ES cell gene targeting experiments are conducted with a Dnmt1 promoter construct combining all three cis elements.

### CROSS-REFERENCE TO RELATED APPLICATIONS

This application claims the benefit of priority to U.S. Provisional Patent Application Ser. No. 61/226,234 filed Jul. 16, 2009 and entitled CONTROL OF ENDOGENOUS DNMT1 GENE EXPRESSION BY EXOGENOUS BINARY REGULATORY SYSTEMS, which is incorporated by reference herein in its entirety.

### STATEMENT REGARDING FEDERALLY-SPONSORED RESEARCH

This invention was made with government support under contract No. R01 CA075090-09 awarded by the National Institutes of Health. The government has certain rights in the invention.

### FIELD OF THE INVENTION

Aspects of the present invention relate generally to regulation of gene expression, and more particularly to novel and efficacious compositions and methods for control of endogenous gene expression, including but not limited to transcriptional control of endogenous genes in mammalian systems using cis/trans binary regulatory DNA sequences.

### BACKGROUND

Several techniques have been developed to probe gene function through either loss or gain of function analysis in mice, such as conventional/conditional knock-outs and transgenics. However, cell lethality has limited the successful implementation of knockout technologies for some genes in, e.g., cells and transgenics. In vivo knockdown approaches relying on tet-regulated SiRNA expression may be able to address this problem for some genes, but SiRNA development has proved challenging for some genes, and the degree of repression by RNA interference varies.

In mammals, DNA methylation is a covalent enzymatic modification occurring at C-5 position of cytosine ring in the context of CpG dinucleotide, cytosine followed by guanine. This reaction is performed by the enzyme called DNA methylatransferase. There exist at least three known functional DNA methyltransferases in mammal, Dnmt1, Dnmt3a, and Dnmt3b (1). Dnmt1 is responsible for maintaining methylation content upon DNA replication based on the methylation information on the parent strand, so it is called maintenance methylatransferase. Dnmt3a and Dnmt3b are called de novo methyltransferase since they can function independently of methylation status of the parent strand (2). CpG dinucleotide is severely underrepresented in most part of mammalian genome due to the spontaneous deamination of methylated cytosine, resulting in C to T transition. However, there are regions called CpG islands, which are rich in CpG content (3). CpG islands are often associated with promoters of genes. About a half of the promoters of the genes in mammal have a CpG island (3). In general, CpG islands are not methylated in normal cells with a few exceptions, such as imprinted genes and genes on inactive X chromosome (2). In cancer cells, however, some CpG islands become methylated, resulting in abnormal gene silencing. Hypermethylated CpG islands are often associated with tumor suppressor genes, suggesting the important role of DNA methylation in tumorigenesis (4). Surprisingly, the silencing of classic tumor suppressor genes by aberrant promoter hypermethylation is at least as common as the disruption of classic tumour suppressor genes by genetic mutation in human cancer (5). However, the mechanisms in which this abnormal promoter hypermethylation occurs still remain largely unknown.

There is, therefore a pronounced need in the art for novel and efficacious compositions and methods for transcriptional control of endogenous genes (e.g., in a mammalian cell or cell system), including but not limited to efficacious methods for control of endogenous gene expression of the major DNA methyltransferase genes (e.g., Dnmt1).

### SUMMARY OF THE INVENTION

Particular aspects provide novel and efficacious methods for control of endogenous gene expression. The inventive methods represent the first demonstration of transcriptional control of an endogenous gene in a mammalian system. In certain aspects, this can be applied to any gene and serve as a useful tool, particularly where conventional conditional knock-out and transgenic approaches cause cell lethality, and/or where SiRNA approaches have failed.

Certain aspects provide efficacious methods for control of endogenous gene expression of the major DNA methyltransferase, Dnmt1 gene, and provide fine-tuned temporo-spatial control of endogenous Dnmt1 gene expression to enable, for example, elucidation and modulation of DNA methylation and other epigenetic mechanisms in cancer.

In particular embodiments, the novel methods comprise a novel transcriptional regulatory system involving three exogenous binary systems, lacI, tetR and Gal4, which allow for the reversible up- and down-regulation of a target gene from its endogenous locus.

In particular aspects, a number of modifications were engineered into both the lac operator and repressor, which significantly improved the repression as compared to wild type (WT) lac and tet systems.

In certain embodiments, lac operator sequences were introduced into the endogenous Dnmt1 promoter through gene-targeting, without significantly altering promoter activity in the absence of repressors. Real-time RT-PCR analysis of targeted ES cells showed that the expression of the targeted allele was substantially reduced by the introduction of lac repressor to less than 10% of WT allele. In particular embodiments, the lacO targeted Dnmt1 allele has been introduced into the mouse germline, to provide a respective transcriptional control system in vivo.

In additional embodiments, two binary systems, tet operator/tetVP16 and Gal4 binding sequence/Gal4VP16 were introduced to achieve upregulation of Dnmt1. In particular aspects, luciferase reporter assays are used in assays to modulate expression from the Dnmt1 promoter over two orders of magnitude, ranging from 3.7% to 800% of unregulated expression by combining these three binary systems.

In further aspects, gene targeting experiments are conducted in mouse ES cells with a Dnmt1 promoter targeting construct that combines the cis elements for all three of these systems.

In certain embodiments, endogenous Dnmt1 expression was successfully up and down regulated in a reversible temporal-spatial manner in mice. In further embodiments, this technology was potent enough to reproduce the embryonic lethal phenotype of genetic knock-out and to attenuate transcription elongation, and the lethal phenotype was rescued by IPTG treatment. This establishes the first paradigm of experimental rescue of an embryonic lethal phenotype in loss-of-function genetics, and enables study of regulated expression for genes for which in vivo characterization have been limited in the prior art due to a lethal phenotype.

Certain aspects provide for controlling eukaryotic (e.g., mammalian) gene expression through the institution of a physical access to the endogenous promoter, and in exemplary embodiments with Dnmt1, a gene for which the use of conventional/conditional knock-outs and transgenic approaches have been limited due to embryonic or cell lethality. This invention, however, is not limited to the Dnmt1 gene, and rather is generally applicable to other genes, other organisms. and other systems.

According to particular embodiments, the current invention relates to a method for transcriptional control of an endogenous gene, comprising: introducing, into a suitable region of a target endogenous mammalian target gene sequence of a mammalian cell having a genome, at least one cis element of at least one exogenous binary regulatory DNA sequence; introducing, into the genome of the mammalian cell, an expression cassette/vector encoding at least one corresponding trans element of the at least one binary regulatory DNA sequence, wherein the at least one cis element is operative with the target gene sequence and the at least one trans element to provide for transcriptional control of the endogenous target gene expression by the at least one binary regulatory DNA sequence. According to additional embodiments, the transcriptional control involves introducing multiple cis elements. According to further embodiments, introducing of the at least one trans element comprises introducing multiple trans elements. According to still further embodiments, the expression cassette/vector encoding the at least one corresponding trans element of the binary regulatory DNA sequence comprises mammalian promoter and/or regulatory sequences.

In certain aspects, the transcriptional control comprises administration of an agent suitable to modulate the intracellular interaction between the cis and trans elements of the binary regulatory DNA sequences. In further aspects, the agent is selected from the group consisting of allolactose, lactose, IPTG, tetracyclines, and galactose. In still further aspects, the cis and trans exogenous binary regulatory DNA sequences are of heterologous origin. In yet further aspects, the cis and trans exogenous binary regulatory DNA sequences are from microorganisms, including bacteria and yeast.

According to certain aspects, the binary regulatory DNA sequences are selected from the group consisting of lac operator/repressor, tet operator/repressor, Gal4 operator/repressor, and functional variants (muteins, fusions, deletions, insertions, fragments, derivatives, etc) thereof. According to further aspects, transcriptional control of the endogenous gene expression by the binary regulatory DNA sequences comprises transcriptional repression or transcriptional activation. According to yet further aspects, introducing at least one cis element of at least one exogenous binary regulatory DNA sequence comprises recombination. In particular aspects, the target gene comprises the Dnmt1 gene. In further aspects the method of the invention comprises: introducing the at least one cis element into at least one cell of a first transgenic mammal; introducing the at least one trans element into at least one cell of a second transgenic mammal crossing the first and second transgenic mammals to provide at least one cell, or provide at least one progeny animal with at least one cell, wherein the at least one cis element is operative with the target gene and the trans element to provide for transcriptional control of the endogenous gene expression in the at least one cell by the at least one binary regulatory DNA sequence.

According to particular embodiments, the current invention includes a mammalian cell, comprising at least one of: an endogenous target gene sequence having suitably inserted therein at least one cis element of at least one exogenous binary regulatory DNA sequence; and an expression cassette/vector, within the genome of the mammalian cell, encoding at least one corresponding trans element of the at least one binary regulatory DNA sequence, wherein the at least one cis element is operative with the target gene sequence and the at least one trans element to provide for transcriptional control of the endogenous target gene expression by the at least one binary regulatory DNA sequence. According to further aspects, the cell is that of, or within a transgenic animal. According to yet further aspects, the cell is an embryonic stem cell. According to still further aspects, the target gene comprises the Dnmt1 gene.

In certain embodiments, a lacI repressor comprises a stabilizing amino acid adjacent to the N-terminal lysine. In further exemplary embodiments, the stabilizing amino acid is, for example, Gly, Ala, or Val. In yet further embodiments, the lacI sequence comprises a consensus GCCACCATGG (SEQ ID NO:1) sequence (or GNCACCATGG (SEQ ID NO:2) sequence, wherein the N is selected from the group consisting of cytosine, guanine, and thymidine.), or comprises the above described stabilizing amino acid in combination with comprising said consensus sequence.

According to particular embodiments, a lacI repressor, comprises at least one mutation selected from the group consisting of proline to tyrosine change at the third amino acid residue and serine to leucine change at the 61^{st} amino acid residue. According to further embodiments, the lacI repressor (or variant, or biologically active portion thereof) is fused to another protein, wherein the other protein is, for example, VP16. Particular embodiments provide a lacI repressor, comprising a fusion protein, wherein the fusion protein is VP16. Particular embodiments provide a lac operator, comprising a symmetric lac operator sequence consisting, for example, of TGTGGAATTGTGAGC-GCTCACAATTCCACA (SEQ ID NO:3).

In certain aspects, the invention involves a method for bidirectional cloning, comprising: generating at least one deletion mutant construct; and inserting at least one binary regulatory DNA sequence at different locations with respect to the at least one deletion mutant construct, wherein only three restriction enzymes (e.g., BsaI, AvrII and SpeI are used, and wherein the generating at least one deletion mutant construct and the inserting at least one regulatory sequence are synchronized. In further aspects, the regulatory sequence is an operator. In still further aspects, the operator is selected from the group consisting of the lac operator, the tet operator, and the Gal4 regulatory sequence.

### BRIEF DESCRIPTION OF THE DRAWINGS

FIG. 1 is a schematic view of the binary transcriptional regulatory system. According to exemplary aspects of the present invention, the this regulatory system is made by crossing a mouse transgenic line expressing either a repressor or activator or both driven by a tissue specific promoter with a mouse line in which the endogenous promoter of a target gene is modified with operator sequences that are controlled by the repressor or activator. This system allows for transcriptional regulation, either up or down expression, upon the binding of activators or repressors to the operators, respectively.

FIG. 2 shows, according to exemplary aspects of the present invention, the first demonstration, using real-time PCR, of repressing an endogenous gene expression with lac operator/repressor.

FIG. 3 indicates the comparison of repression capacity between Lac and Tet repressors. Panel A shows the reporter and repressor constructs used in the transient report assay; non-functional lacI (NFlacI), wild type lacI (Lad), lacI with tight binding mutation (LacIY), wild type tet repressor (TetR), tet repressor with nuclear localization signal (tetRNLS), lac operator (O), and tet operator (T). Panel B indicates the activities of the reporter genes in the absence of repressors; CMV-O and CMV-T is the reporter containing the lacO or the tetO. Panel C shows the reporter activities in the presence of repressors as indicated.

FIG. 4 shows the comparison of repression capacity between Lac and Tet repressors. Panel A shows the reporter and repressor constructs used in the transient report assay. Panel B indicates the repression of a promoter construct having a lacO (O) followed by tetO (T). Panel C shows the repression of a promoter construct having a tetO (T) followed by a lacO (O).

FIG. 5 shows, according to exemplary aspects of the present invention, inducible and reversible up and down endogenous gene regulation.

FIG. 6 shows, according to exemplary aspects of the present invention, the repressing effect of tight binding mutations.

FIG. 7 shows, according to exemplary aspects of the present invention, exemplary stabilizing amino acid insertions for Lac repressor (SEQ ID NOS:5 and 6). Panel A (top) shows the coding sequence for the particular amino acids that were inserted in between the first codon (ATG) and the second codon (AAA). Panel A (bottom) shows the reporter assay using the stabilized Lac repressor. Panel B (top) shows the experimental scheme for the quantification assay for the repressors and (bottom) the results from the scheme.

FIG. 8 shows, according to exemplary aspects of the present invention, the effect of stabilized lac repressors (SEQ ID NOS:7-9) in a reporter assay.

FIG. 9 indicates, according to exemplary aspects of the present invention, the results of consensus (O), symmetrical (S), or double consensus (O) operators in a luciferase reporter assay. Panel A shows the sequence of the consensus (O) (SEQ ID NO:10) and symmetrical operators (S) (SEQ ID NO:3) used in the reporter assays. Panel B shows the luciferase value from reports harboring either the consensus (O) or symmetrical operator (S). Panel C shows the luciferase activity from reporter constructs harboring either one (O) or two (O) operators.

FIG. 10 shows, according to exemplary aspects of the present invention, the reversible control of the modified Dnmt1 promoter using IPTG. Panel A (top) shows the reporter construct and the repressors that were transfected into cells and (bottom) the expression level from the resulting transfection either in the presence or absence of IPTG. Panel B shows the expression level from the reporter construct using the indicated repressor construct and the indicated IPTG concentration.

FIG. 11 shows the exemplary organization of alternative promoters and Dnmt1 exons and genomic DNA and mRNA in somatic cells (1s), pachytene spermatiocytes (1 p) and oocytes (10). From Mertineit et al., (1998) Development 125, 889.

FIG. 12 shows, according to exemplary aspects of the present invention, the characterization of the Dnmt1 promoter. Panel A shows (top) the genomic configuration of Dnmt1 locus, including the oocyte (10), somatic (1s), and pachytene (1p) exon 1. In addition, FIG. 12, panel A shows the sequence homology comparison with rat and human Dnmt1 promoter sequences and transcription factor binding site search (TF search). Panel A (bottom) demonstrates, via reporter assays, the expression of the Dnmt1 promoter deletion mutants. Panel B shows, by reporter assays, the expression of 3′ and internal deletion mutants of the Dnmt1 promoter.

FIG. 13 shows, according to exemplary aspects of the present invention, the effect of modifying the Dnmt1 promoter with lacOs. Panel A shows the expression from the Dnmt1 promoter modified with lac operator without the lac repressor present. Panel B is a schematic of the Dnmt1 promoter with different forms of the lac operator: symmetric operators (S) and double operators (O).

FIG. 14 shows, according to exemplary aspects of the present invention, the effect of modifying other promoters with the lac operator sequence. Panel A (top) shows the human ubiquitin C promoter (hUbc) and SV40 promoter as modified with two copies of lac operators and (bottom) the level of expression from these modified promoters. Panel B (left) shows the rabbit beta-globin intron as modified with the symmetric operators. This modified intron was placed between the Dnmt1 promoter and the luciferase reporter. On the right, the expression levels from the Dnmt1 promoter-modified intron-luciferase reporter construct is shown.

FIGS. 15A-G show, according to exemplary aspects of the present invention, the sequence of the targeting vector for Dnmt1 promoter that has been modified with lacO, tetO, gal4 binding sequences (SEQ ID NO:11).

FIG. 16 shows, according to exemplary aspects of the present invention, the sequence (SEQ ID NO:12) of the modified lacI with the stabilizing amino acid (GGC), and the tight binding mutation (TAT) labeled in red and blue, respectively.

FIG. 17 shows, according to exemplary aspects of the present invention, a schematic of the bi-directional cloning strategy.

### DETAILED DESCRIPTION OF THE INVENTION

The ability to control gene expression in living organisms through genetic manipulations has been essential in advancing our understanding of physiologic and pathologic processes of biology. Genetic tools in functional genomics can be broadly categorized into two types; loss-of-function and gain-of-function approaches. Knock-out technology via gene-targeting (or targeted transgenesis) has been arguably the most successful application of the first category. Gene-targeting allows for the inactivation of a target gene by introducing a genetic modification(s) at a predetermined site(s) in the genome through homologous recombination that results in a null mutation (Thomas and Capecchi, 1986). In contrast to gene-targeting, the transgenic approach (or random transgenesis) enables the integration of foreign DNA into genomic sites that are not known a priori (Ristevski, 2005). The random integration is achieved through injection of transgenes into the male pronucleus of a fertilized egg or transfection of transgenic constructs into embryonic stem (ES) cells. This random transgenesis, in general, has been used to study gene function through elevation of gene expression.

Both targeted and random transgenesis have produced numerous animal models that provide valuable functional analyses for many genes and insights into mechanisms of various biological processes as well as diseases. However, the disadvantage of these approaches is that genetic alterations caused by these approaches are germline mutations that are present in all cell types at all time. This has limited the broad application of these technologies. For example, germline null mutations often cause a lethal phenotype, which prevents the functional analysis of the gene of interest in adult animals. Furthermore, universal gene activation or inactivation may be subject to a pleiotropic effect that may complicate the interpretation of outcomes. To overcome these limitations and to achieve a more precise gene expression control, conditional gene-targeting and inducible transgenics have been developed (Chien, 1996; Metzger et al., 1995; Glaser et al., 2005; Gossen and Bujard, 1992).

The study of the role of Dnmt1 in tumorigenesis has been limited because of the non-viability of mice lacking functional Dnmt1. One alternative approach to overcome this problem is to provide a conditionally inactivated allele of Dmnt1, in which Dnmt1 will be inactivated in time and/or tissue specific manner.

The most commonly used system to generate conditionally inactivated allele is the Cre-loxP system, in which Cre-mediated deletion of a target gene occurs in tissue specific manner depending upon the promoters driving Cre recombinase expression (6). A conditionally inactivated allele of the mouse Dnmt1 has been successfully generated using Cre-loxP system (7, 8). However, this system turned out to be unsuitable for the study of the role of Dnmt1 in early stages of cancer development in vivo with two reasons. First, differentiated cells that lose functional Dnmt1 expression undergo p53-dependent cell death (7). Second, Cre-based recombination is a highly stochastic event, that is, in this system some cells undergo Cre-mediated deletion of Dnmt1 and subject to p53 dependent cell death, but some other cells do not go through Cre-based deletion of Dnmt1 and have normal level of Dnmt1 expression. An ideal system to study the role of DNA methylation in tumorigenesis might be a system in which all of the targeted cells have partially inactivated Dnmt1 expression level that do not cause either embryonic lethality or p53-dependant cell death, instead of a full inactivation. To accomplish this, particular aspects provide an in vivo binary transcriptional repression system based on a prokaryotic gene switch system, lac operon similar to the widely used binary transcriptional activation systems, tet operon (9, 10, 11). According to particular aspects, the Lac operator/repressor system has a couple of features that make it better suited for the present purposes as compared to other prokaryotic operator/repressor systems. First, lac repressor works as a tetramer, and this tetramer can bind to two lac operators on one DNA stretch and induce the intervening DNA to form a loop (12). The distance between two operators can be more than 500 bp. This feature assists to insert lac operator sequences into Dnmt1 promoter at locations that do not interfere with promoter function in the absence of the repressor, yet result in good repression in the presence of the repressor. According to additional aspects, with other prokaryotic operator/repressor systems whose repression mechanism relies only on steric hindrance (13), it may not be possible to insert operator sequences in eukaryotic promoters at locations that do not impede promoter function in the absence of the repressor, yet result in good repression in the presence of the repressor, because eukaryotic promoters have evolved in the absence of operator/repressor based transcriptional regulation system, whereas the prokaryotic promoters controlled by the operon systems have evolved in a way that they can accommodate operator sequences in vicinity of transcriptionally critical region. Second, the lac operator/repressor system has the longest operator sequence among prokaryotic operator/repressor systems, which reduces the probability of the existence of fortuitous lac operator sequences in the mouse genome. In particular aspects, the Dnmt1 promoter is modified with lac operators and transgenic mice are generated that express lac repressor in limited tissues, using tissue-specific promoters, and in particular aspects of this system the results are partial inactivation of Dnmt1 expression, not full inactivation. First, prokaryotic operator/repressor systems have been shown to be leaky in mammalian cells (14, 15, 16). Second, by generating several lines of lacI transgenic mice with different copy numbers and integrated sites, varying degrees of expression levels of lac repressor, resulting in a range of repression capacities are provided.

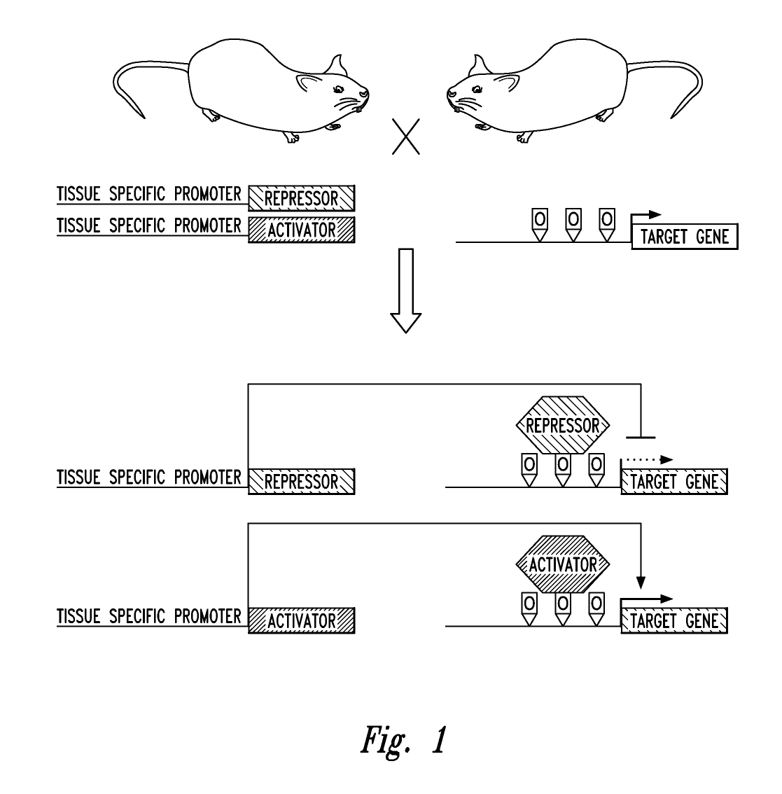

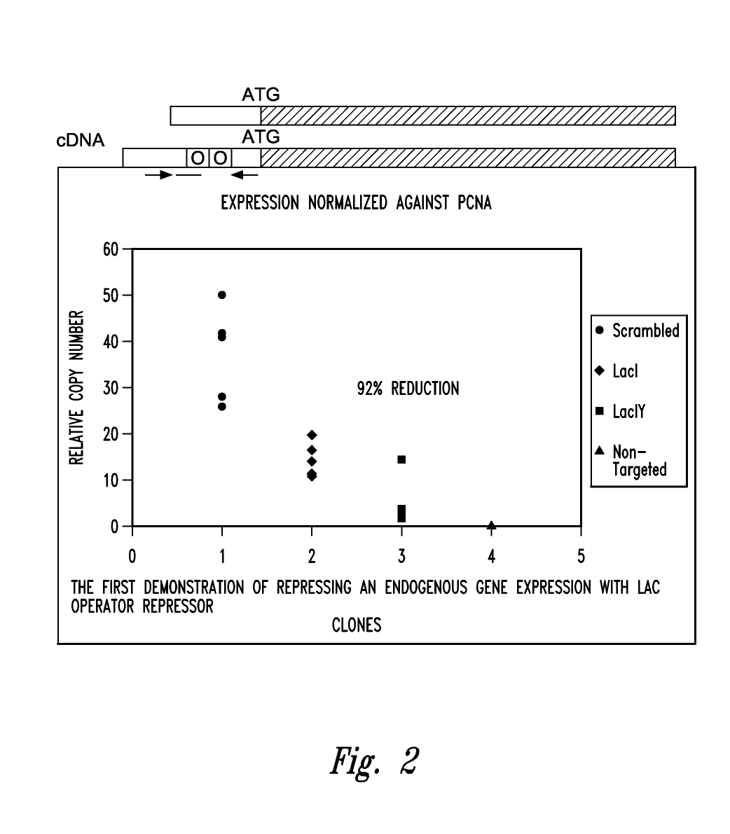

Particular embodiments provide for application of the repression and activation binary systems to an exemplarly endogenous gene, Dnmt1, such that cis-elements of the binary systems were integrated into the endogenous promoter of the target gene, and the repressor and transactivator induce repression and activation through a direct interaction with the transcription machinery on the promoter (FIG. 1).

### Terms Used Herein

As used herein, an endogenous gene is one that originated from within a particular organism, tissue, or cell.

As used herein, exogenous binary regulatory DNA sequence are sequences that originated from another gene, or from outside a particular organism, tissue, or cell and is suitable for controlling the regulation of a target endogenous gene expression within a particular organism, tissue, or cell.

As used herein, an expression cassette/vector is a nucleic acid sequence that may constitutively express or inducibly express a certain protein. In certain embodiments, the expression cassette/vector as used herein encodes for a transcriptional regulator, and is operatively driven by a promoter that is active in the host cell.

As used herein, a cis element is a nucleic acid sequence that is operably linked to the gene of interest and suitable for binding trans elements, providing for altered transcriptional expression of a certain target gene operably linked to the cis element.

As used herein, a trans element is an regulatory element, which can be DNA, RNA, or protein, suitable for binding to cis elements to alter transcriptional expression of a certain gene.

As used herein, DNA or nucleic acid sequences that are referred to as heterologous origin are those DNA or nucleic acid sequences derived from an organismal source other than the organism in which it is to be or has been inserted.

As used herein, microorganisms, may refer to any microorganism and includes, but is not limited to bacteria and yeast.

As used herein, regulatory sequences include but are not limited to cis elements, such as those examples described herein for illustrative purposes.

### Biologically Active Variants

Functional variants of the repressors and activators described herein can be naturally or non-naturally occurring. Naturally occurring variants (e.g., polymorphisms) are found in humans or other species and comprise amino acid sequences which are substantially identical to the amino acid sequences of the repressors and activators as disclosed herein.

Non-naturally occurring variants which retain substantially the same biological activities as naturally occurring protein variants, are also included here. Preferably, naturally or non-naturally occurring variants have amino acid sequences which are at least 85%, 90%, or 95% identical to the amino acid sequences of the repressors and activators as disclosed herein. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 2:

TABLE 2
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant.

Variants of the repressors and activators described herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (see, e.g., Mark et al., U.S. Pat. No. 4,959,314).

Preferably, amino acid changes in the repressors and activators described herein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant.

Truncations or deletions of regions which do not affect functional activity of the proteins are also variants of the repressors and activators described herein. Covalent variants can be prepared by linking functionalities to groups which are found in the amino acid chain or at the N- or C-terminal residue, as is known in the art.

It will be recognized in the art that some amino acid sequences of the repressors and activators described herein of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors (Ostade et al., Nature 361:266-268, 1993). Thus, the repressors and activators described herein of the present invention may include one or more amino acid substitutions, deletions or additions, either from natural mutations or human manipulation.

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic (see, e.g., Pinckard et al., Clin. Exp. Immunol. 2:331-340 (1967); Robbins et al., Diabetes 36:838-845 (1987); and Cleland et al., Crit. Rev. Therapeutic Drug Carrier Systems 10:307-377 (1993)).

Amino acids in the repressors and activators described herein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given repressor and activator as described herein will not be more than 50, 40, 30, 25, 20, 15, 10, 5 or 3.

In addition, pegylation of the repressors and activators as described herein and/or muteins is expected to provide such improved properties as increased half-life, solubility, and protease resistance. Pegylation is well known in the art.

### Fusion Proteins

Fusion proteins comprising proteins or polypeptide fragments of the repressors and activators as described herein can also be constructed. Fusion proteins are useful for generating antibodies against amino acid sequences and for use in various targeting and assay systems. For example, fusion proteins can be used to increase the repressors and activators described herein of the invention interaction with DNA or which alter their biological function. Fusion proteins comprising a signal sequence can be used.

A fusion protein comprises two protein segments fused together by means of a peptide bond. Amino acid sequences for use in fusion proteins of the invention can be utilize the amino acid sequences of the repressors and activators as disclosed herein or can be prepared from biologically active variants of those repressors and activators, such as those described above. The first protein segment can include the repressors and activators as described herein.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) VP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding region for the protein sequence of the repressors and activators as disclosed herein in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Example 1

### Materials and Methods

Plasmid Construction

The pGL3O and pGL3T plasmids were generated by inserting DNA oligos containing a lac operator and a tet operator flanked by a SacI and HindIII sites (5′-3′) into the pGL3 Basic (promega) vector using the enzyme sites.

The luciferase reporter plasmid harboring the CMV promoter (pCMVL) was constructed by PCR amplifying the promoter using the forward and reverse primers harboring a NotI and AvrII site respectively. The amplicon was digested with the NotI and AvrII and ligated into the pGL3O vector digested with the NotI and SpeI.

The pCMVOL and pCMVT were generated by PCR amplifying the CMV promoter using the same primers used to generated pCMVL. The amplicon was digested with the NotI and AvrII and ligated into the pGL3O vector digested with the NotI and AvrII.

The pLacI was constructed by replacing the 5′ end (36 bp) and EcoRV-PvuII flanked 150-bp regions of the pSYNIacI (from Dr. Scrable) with the same regions from the wild type lacI. This was done by PCR amplifying the 5′ region of wild type lacI using primers harboring a BamHI (forward) and an XbaI (reverse) sites the 150-bp region using primers containing EcoRI and PvuII sites and inserting the amplicons into the pSYNIacI using the enzyme sites.

The pNFlacI was generated by inserting the lacI in reverse orientation (Jie Wei). The pLacIY was made by replacing the third amino acid (proline) of the lacI with a tyrosine. This was done by PCR using a forward primer harboring a BamHI site and a coding sequence for tyrosine and a reverse primer with an XbaI (reverse) site. The PCR amplicon was inserted into the pLacI using the BamHI and XbaI sites in the amplicon and the vector.

The pTetR and pTetRNLS were generated by PCR amplifying the tetR from the pcDNA6 vector using a forward primer containing a BamHI site and a reverse primer containing an MluI site with and without NLS. The amplicons were inserted into the pLacI vector using the BamHI and MluI sites, which replaces the lacI with tetR or tetRNLS.

The pLacIGY was generated by inserting a glycine residue into the lacIY at a position between the first amino acid (methionine) and the second amino acid (lysine). This was done by PCR amplifying the lacIY using a forward primer harboring a coding sequence of a glycine and a BamHI sites and a reverse primer containing an XbaI site. The PCR amplicon was inserted into pLacIY using the sites.

All the nested deletion constructs and lac operator containing constructs were generated using the same strategy described in Example 4.

The pDL-IN was generated by PCR amplifying the rabbit beta-globin intron from the K14-BG-PL-Rev vector using a forward primer containing a SacI site and a reverse primer with an MluI site. The amplicon was inserted into pDL5 using the same sites.

The phUBCOOL and the pSV4000L was constructed by PCR amplifying the promoters using the forward and reverse primers harboring a NotI and SpeI site respectively and ligating it into the pGL300 using the enzyme sites.

Luciferase Reporter Assay

NIH3T3 cell line was cultured in DMEM with 10% fetal bovine serum (FBS). The empty vector was used as control for transfections of all reporter plasmids. Each experiment was done in at least three biological replicates. The results shown are the means of triplicate points. The Dual Luciferase Reporter Assay Kit (Promega) was used to determine luciferase expression, as a measure for promoter activity. After applying 40 μl of passive lysis buffer, firefly and renilla luciferase expression was measured using a TD-20/20 tube luminometer (Turner BioSystems) or the automated luminometer. 30 μl of firefly substrate was injected in each well. Light intensity was measured. Then 30 μl Stop&Glow renilla substrate was added and luminescence was measured again. The ratio of firefly over renilla luminescence intensity was calculated and used as a measure for promoter activity.

Western Blot Analysis

Whole cell extracts were prepared from NIH3T3 cells at 2, 4, and 6 days after transfection using RIPA buffer. Equal amounts of protein from whole cell extracts were separated on gradient (4-12%) polyacrylamide gels (Invitrogen, Burlington, ON) and then transferred to polyvinylidene difluoride (PVDF) membranes (Bio-Rad, Hercules, Calif.). Blots were probed with the anti-lacI antibodies (Millipore, Temacula, Calif.) for lacI, and anti-Flag antibodies (Millipore, Temacula, Calif.) for Dnmt3b4 followed by incubation with species specific horseradish peroxidase-conjugated secondary antibodies (Santa Cruz, Santa Cruz, Calif.). Proteins were visualized using immobilon Western HPR Substrate (Millipore, Temacula, Calif.).

### Example 2

### Comparison Between lac and tet Control Systems

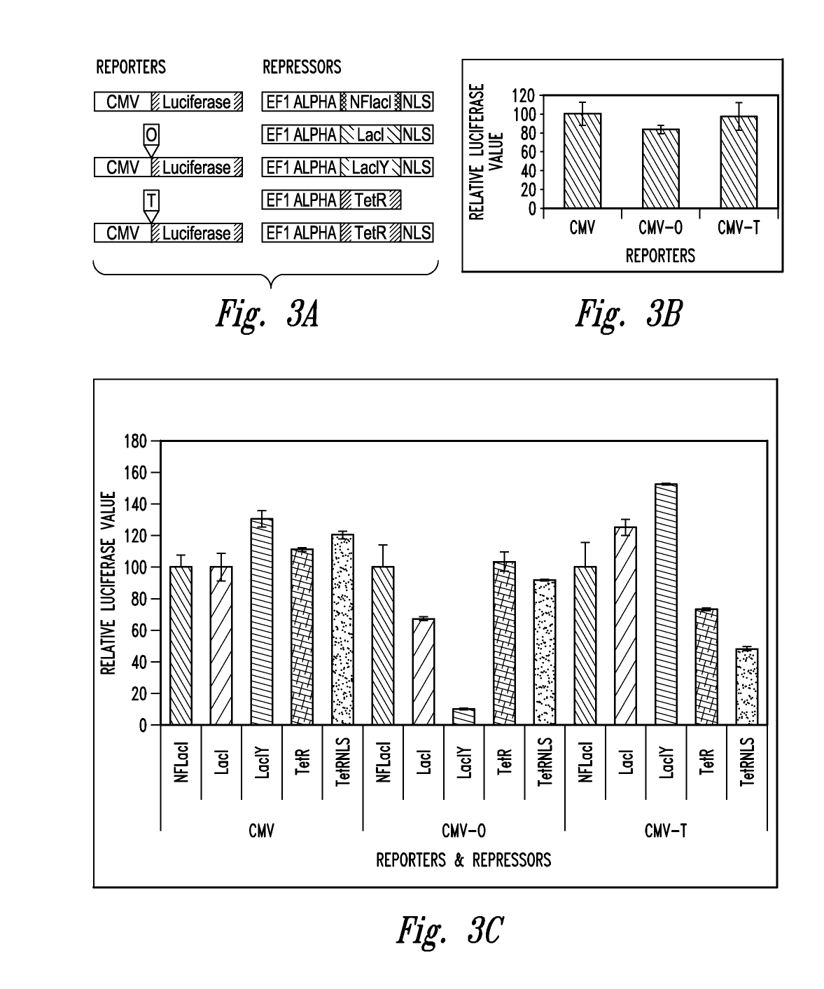

According to certain aspects, to achieve the reversible control of a target gene expression in mammalians, two art-recognized prokaryotic binary transcriptional regulatory systems, lac and tet operator/repressor, were adopted. The functionality and reversibility of both systems for transgenes have been previously demonstrated in mice (Kistner et al., 1996)(Cronin et al., 2001). The Applicants compared the two systems in a luciferase reporter assay. For repression comparisons, the Lac operator (lacO) or Tet operator (tetO) sequence was placed in between the CMV promoter and the luciferase gene. Plasmids harboring repressors are engineered identically except for the region encoding each repressor, thereby allowing the comparison of their repression capacity per gene copy, which is a relevant comparison for a transgenic approach (FIG. 3, panel A). Five different repressors were subjected to the comparison: non-functional lac repressor (NFLacI), wild type lac repressor (LacI), lac repressor with tight binding mutation (LacIY), tet repressor (TetR), and tet repressor with nuclear localization signal (TetRNLS). NFlacI was constructed by inserting the lacI in reverse orientation to make it non-functional. LacIY was generated by adopting a tight binding mutation identified from E. coli that was shown to enhance binding affinity of the repressor to the operator, as disclosed herein (Kolkhof et al., 1992). The same nuclear localization signal used for the lac repressors was engineered into the tet repressor to produce tetRNLS. LacO or tetO insertion alone had a modest deleterious effect on the promoter in the absence of repressors (FIG. 3, panel B). The reporters and repressors were transiently introduced into the NIH3T3 cells in a 1:1 molar ratio. All of the repressors, when compared to NFlacI, showed repression of the CMV promoter at the given conditions (FIG. 3, panel C).

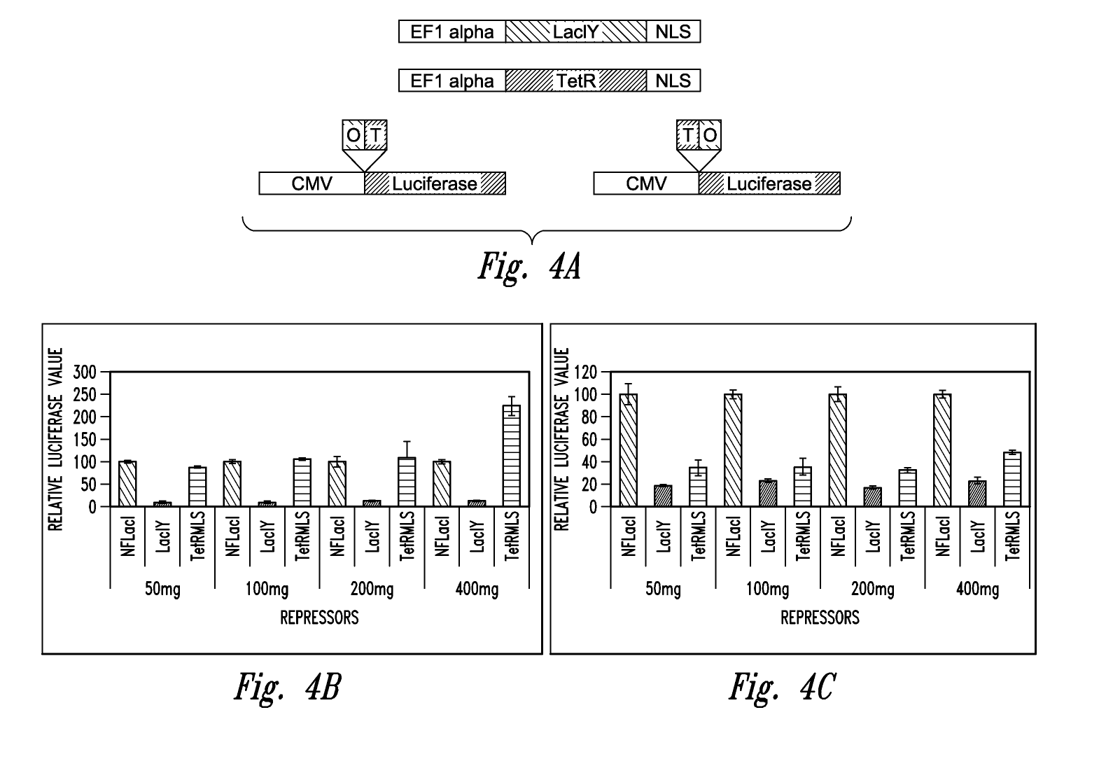

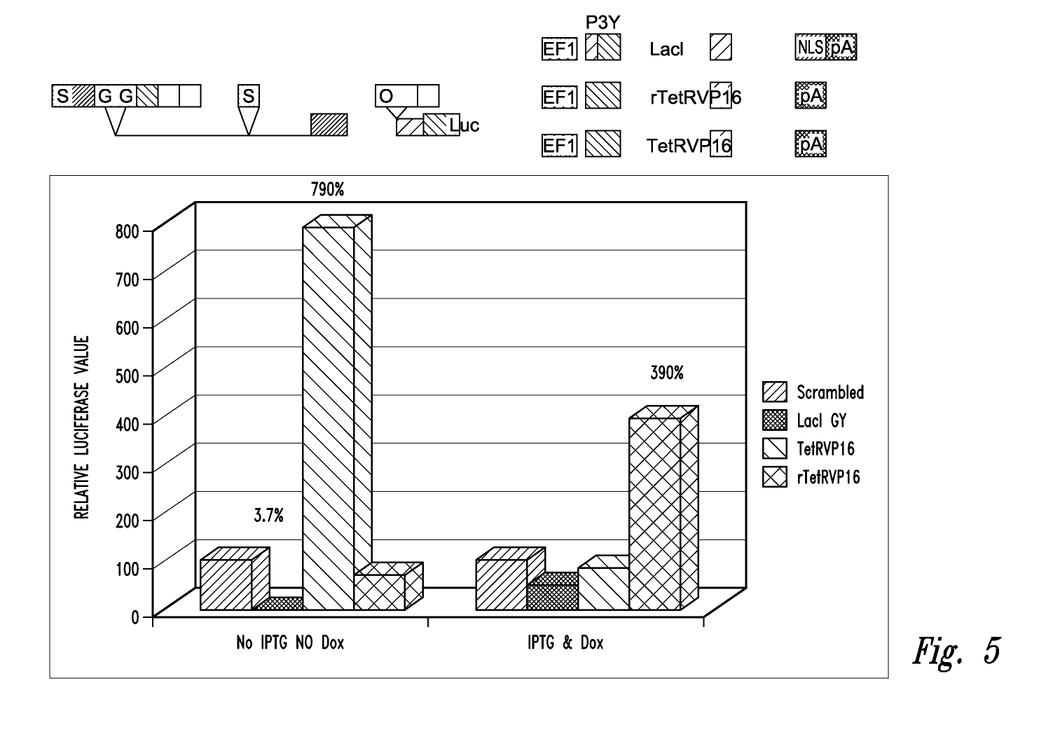

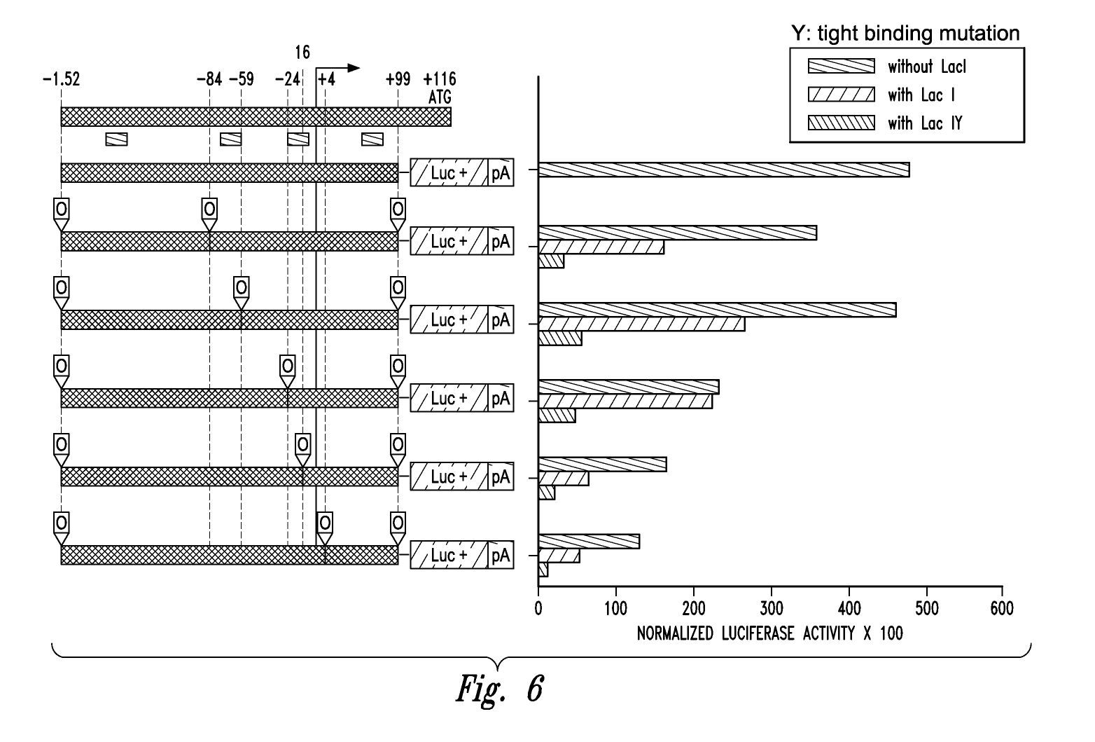

According to additional aspects, to further analyze the lac and tet repressor systems, the repressors are tested on two different reporters separately and side by side in different orders (FIG. 4, panel A). LacIY and tetRNLS were used for the further comparison. Both lac and tet repressors repressed the CMV promoter much more effectively when the corresponding operators were located immediately downstream of the promoter (FIG. 4, panels B and C). However, the repression by tetRNLS was almost completely abolished when the tet operator was located after the lac operator, indicating that the position of the operator is critical for repression by the tet repressor. This observation is consistent with the reliance of the prokaryotic repression mechanism on steric hindrance (Schmitz and Galas, 1979)(Schmitz, 1981). The position of the operator has to be optimal for the repressors to efficiently physically interfere with transcription machinery. However, the repression by lacIY was far less dependent upon the position of the operator. One explanation might be that the tetramerized lac repressor is still able to physically obstruct the transcription machinery even when the operator is positioned further downstream of the promoter. Another possible explanation is that the binding affinity of lacIY is strong enough to block transcription elongation.

### Example 3

### Construction of Symmetric and Consensus Sequence Operators

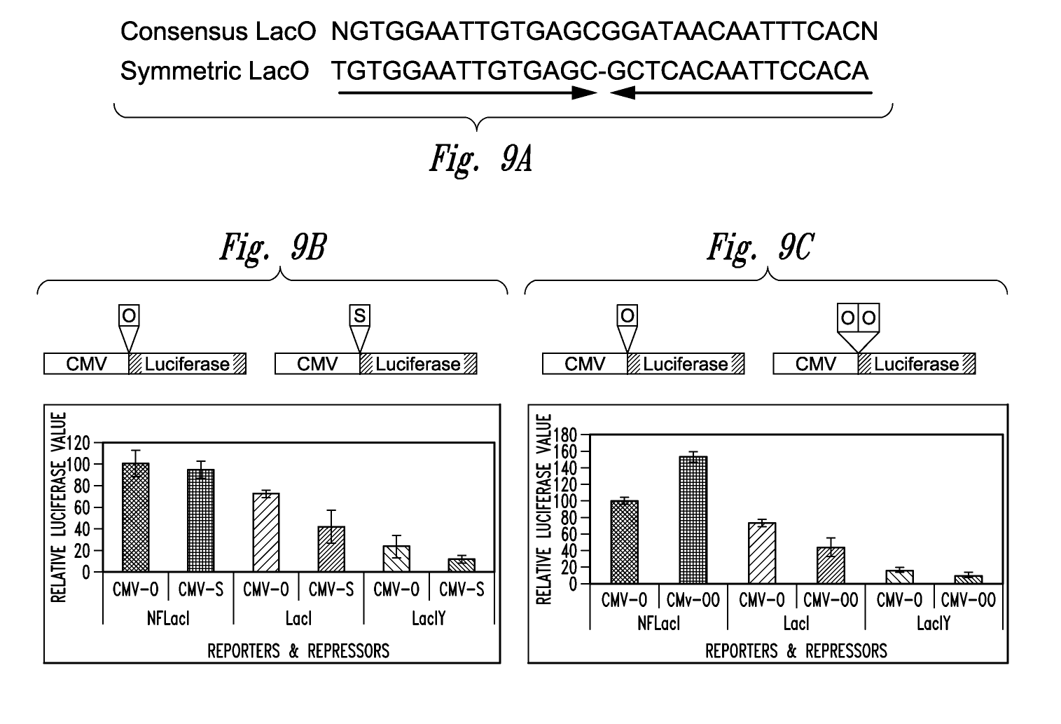

The consensus operator is a 29-bp sequence, which is almost a palindrome, but asymmetric by 4 mis-pairs. A 30-bp perfectly symmetric lac operator sequence (SEQ ID NO:3) has been synthesized and shown to interact with lac repressor at least 10 times more strongly than the consensus sequence (FIG. 9, panel A and (Simons et al., 1984)). Applicants used this modified lac operator to the system to improve the binding affinity between the operator and the repressor. The reporter with a symmetric operator sequence was better repressed by both lacI and lacIY than the one with a consensus operator in the given experimental condition (FIG. 9, panel B). Applicants' also doubled the number of operator sequences to improve repression. In certain embodiments, this alteration improves the binding kinetics between the operator and the repressor. Results from doubling the operator sequence show that repression was improved about 50%, when compared to those sequences harboring only one operator sequence (FIG. 9, panel C).

### Example 4

### Development of a Bidirectional Cloning Strategy

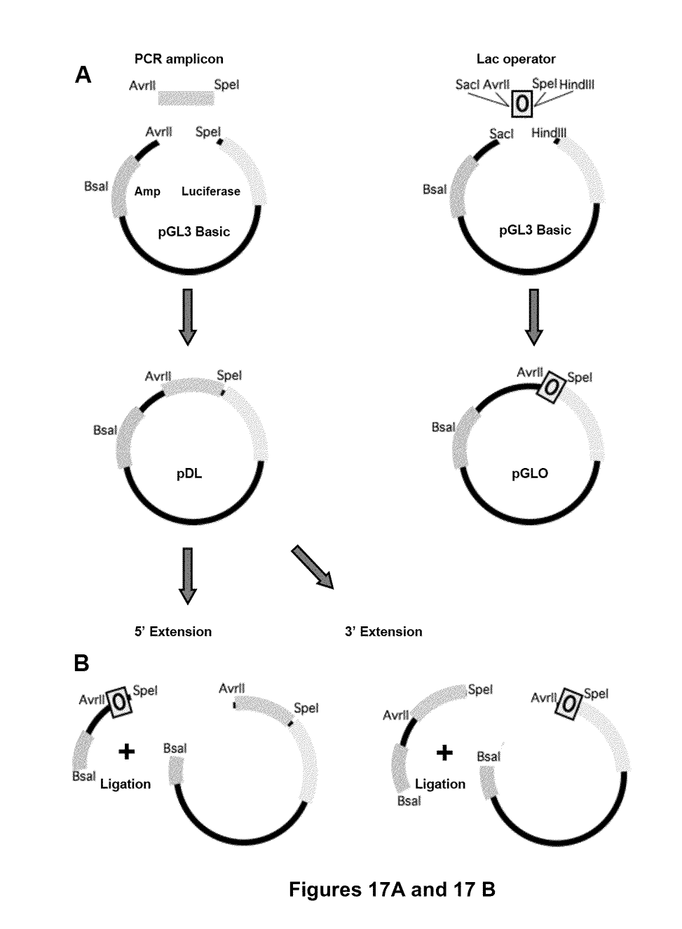

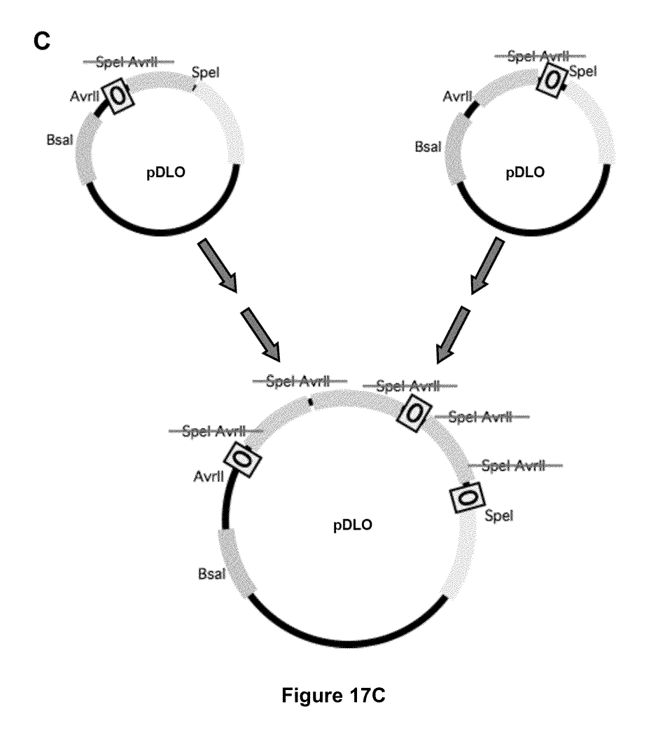

Promoter characterization and operator insertion procedures can be highly time-consuming and labor-intensive due to a large cloning burden related partially to an inability to predict the location and the number of operator insertion sites for a given promoter. This makes it difficult to design the two procedures coherent with regard to the use of restriction sites for generating deletion mutants and operator sequences for each insertion site. To overcome this Applicants' devised a novel cloning method, the bidirectional cloning strategy, which allowed us to generate all the deletion mutant constructs and to insert the operators at different locations using only three restriction enzymes (FIG. 17). This method greatly accelerated the time-consuming and labor-intensive procedures by simplifying and synchronizing the two steps, and is applicable to any other promoter.

More specifically, the bi-directional cloning strategy is accomplished by cloning PCR amplicons into pGL3 Basic Vector using AvrII and SpeI sites. All PCR amplicons were generated to harbor sites at the 5′ and 3′ ends, respectively. These PCR amplicons were then digested with AvrII and SpeI and inserted into pGL3 Basic vector using AvrII and SpeI sites, which resulted in pDL. (FIG. 17, panel A, left). AvrII and SpeI sites were also inserted into pGL3 Basic Vector via a lac operator containing these two sites, resulting in pGLO. (FIG. 17, panel A, right). All PCR amplicons in pDL vector can be modified in both 5′ and 3′ directions. For 5′ extensions, pDL is digested with AvrII and BsaI and ligated with a fragment digested with SpeI and BsaI that contains an insert to be added. For 3′ extension, pDL is digested with SpeI and BsaI and the excised fragment ligated with a pGLO vector digested with AvrII and BsaI. Ligation between AvrII and SpeI destroys both sites, in the final construct, the outer AvrII and SpeI sites are still unique and can be used for further construction. (FIG. 17, panel B). Both 5′ and 3′ extensions can be repeated unlimitedly, resulting in multiple copies of a given sequence or introducing an entirely new sequence. (FIG. 17, panel C). This procedure can be applied to any promoter using a pair of comparable restriction sites absent in the given promoter sequence and a third restriction site unique in the backbone plasmid.

### Example 5

### Development of a Conditional Regulatory System of Dnmt1 Gene Expression Based on lac Operator/Repressor System In Vitro

Experimental Design and Results. In particular aspects, a binary transcriptional regulation system based on the E. coli lacO/lacI system (14, 17, 18) is provided. In certain aspects, the development of a binary transcriptional repression system comprises the following two parts: (1) Modification of Dnmt1 promoter with lacO insertion; and (2) Modification of lacI gene.

The detailed experimental steps are the followings:

5.1 Characterization of the Dnmt1 promoter.
5.2 Test of the effects of lacO insertion with and without lac repressor in a reporter system.
5.3 Insertion of multiple copies of the lacO sequence into the endogenous Dnmt1 promoter using gene targeting.
5.4 Analysis of the effects of the targeted lacO insertion on endogenous Dnmt1 in the presence and absence of lac repressor in cell culture.

### 5.1 Characterization of the Dnmt1 Promoter.

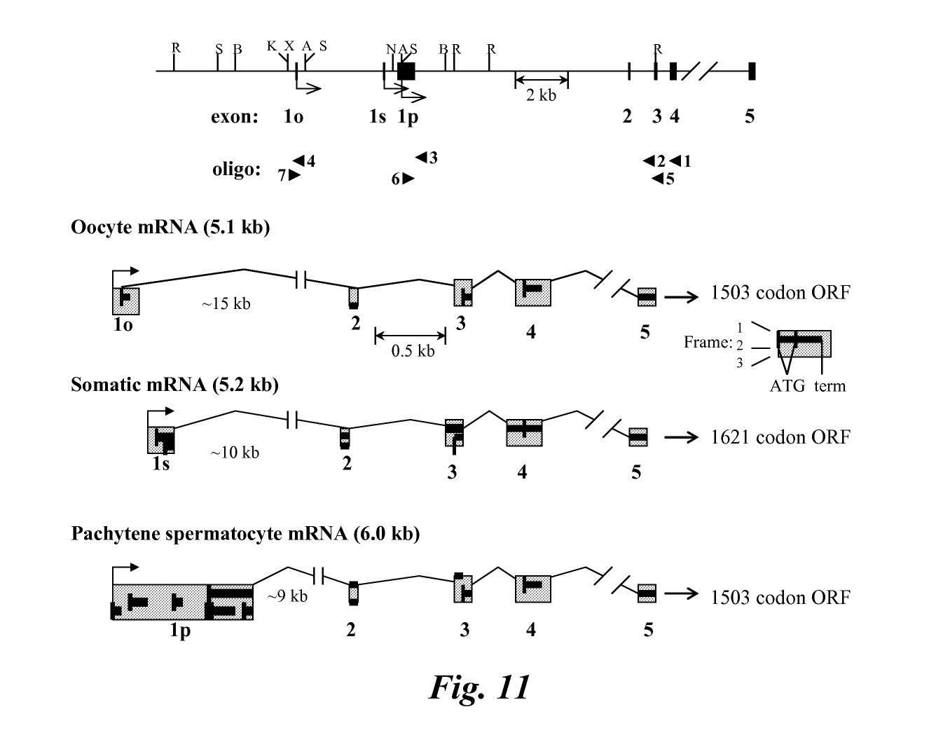

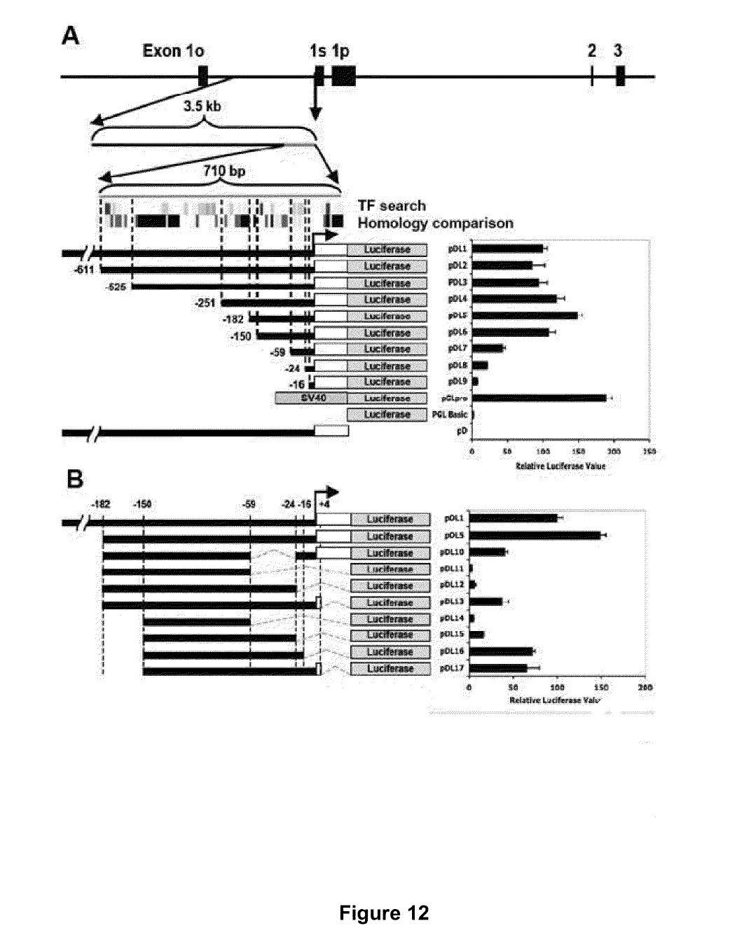

In particular aspects, identification of the locations in Dnmt1 promoter where insertions of the lac operator sequences can be made without damaging promoter function are determined. This requires an extensive characterization of Dnmt1 promoter. In the mouse genome, there are three Dnmt1 promoters two of which are alternative promoters used in oocytes (exon 1o) and in pachytene spermatocytes (exon 1 p) (19, 20). The somatic promoter is located in between these two other promoters as drawn in FIG. 11. Particular aspects comprise modification of the somatic promoter located upstream of Exon 1s since this is the promoter used in most tissues to drive Dnmt1 expression (FIG. 12). In additional aspects, the DNA fragment located immediately upstream of Exon1s is subcloned into a luciferase vector and subsequently analyze for promoter activity in both undifferentiated embryonic stem cells and in NIH/3T3 fibroblasts, using transient luciferase reporter assays. In further aspects, nested deletion constructs and internal deletion constructs are generated to identify putative transcriptional elements, based on two computer based information, transcriptional binding factor search and the sequence homology with other two species (Human and Rodent).

Results. A 3,461-bp MspI fragment derived from a genomic phage library constructed with 129/SvJae mouse genomic DNA was subcloned. This MspI fragment extends from 3,418 by upstream of the transcription initiation site to 99 by downstream of the transcription initiation site (FIG. 12, panel A). The 3′ end of this fragment is located 17 by upstream of the translation initiation site. This fragment was cloned into a luciferase reporter plasmid and tested the promoter activity in NIH/3T3 cells. This fragment showed about a half of promoter activity as compared to SV40 promoter in our transient transfection assay. A series of 5′, 3′, and internal deletion mutants of the promoter were generated, and those mutants tested in reporter system. All of the deletion mutants were designed such that each break point in the promoter does not reside within either the putative transcription factor binding sites or the regions with high homology amongst the three species so that those break points can be used for operator insertion without breaking putative cis-elements.

Increasing promoter activity was observed as the promoter was deleted from the 5′ end to −182 by upstream of the TSS, suggesting the presence of repressor binding site(s) within the deleted regions (FIG. 12, panel A). On the other hand, as the promoter was further deleted from −182 to −16 by upstream of the TSS, promoter activity gradually decreased by several fold. This result suggests that the region (−182 to −16 bp) contains several important cis-elements as reported previously (Kishikawa et al., 2002)(Kimura et al., 2003). Some of the putative transcriptional cis-elements have been further characterized. Results from the 3′ and internal deletion constructs confirmed the observations from the 5′ deletion mutants (FIG. 12, panel B).

5.2 Test of the Effects of lacO Insertion with and without Lac Repressor in a Reporter System.

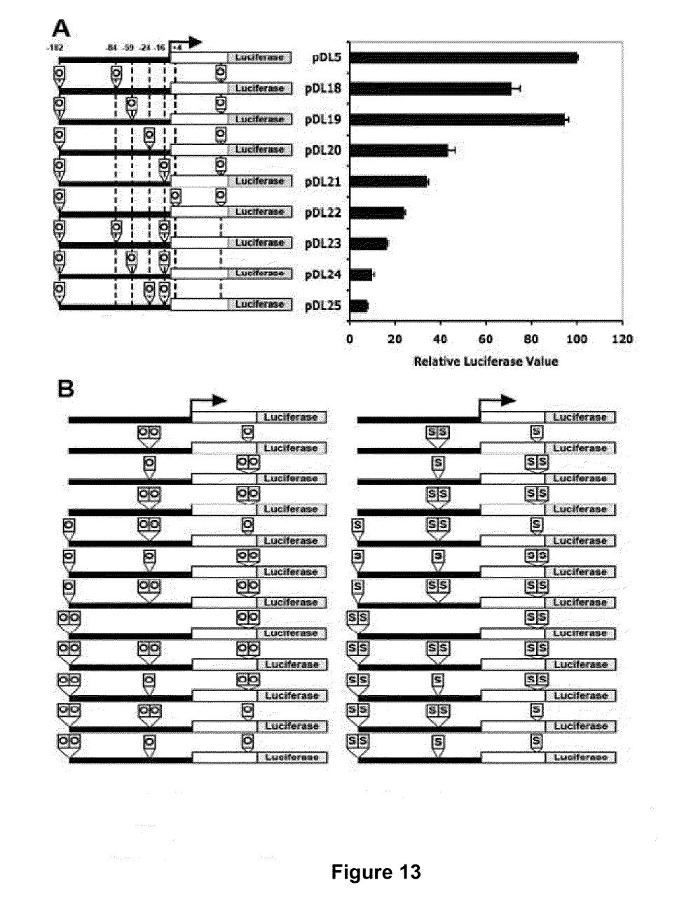

The effects of lacO insertion with and without lac repressor in a reporter system are tested (FIG. 13, panel A). As stated above, a system is desired that will allow regulation of Dnmt1 expression only in a desired specific tissue and at a specific time. This comprises identification of locations where lacO insertion has no effect on the promoter activity. With identified lacO insertion sites that do not affect expression in either of the two tested cell types, various combinations of lacO insertion at multiple locations are tested to determine the number of locations to insert lacO sequences to get the best repression with transiently transfected lac repressor.

Based on Applicants reporter assay data with deletion mutants, several locations for lacO insertion were chosen around the putative transcriptional cis-elements. For lacO insertion, a bidirectional cloning strategy (See Example 4) was developed that allowed inserting lacO sequences at different locations using the same pair of compatible restriction enzymes (SpeI and AvrII) without involving screening steps. With this strategy, more than 200 constructs were generated with different numbers of lacO sequences at different locations with a homogeneous sequence at each junction in relatively short time. The effect of lacO insertion at each individual site on the promoter activity was tested with and without lac repressor, and three sites (−182, −59, and +99) were identified that can accommodate lacO insertion with almost no negative effect on promoter activity. The lacO sequences were then inserted at multiple locations to determine the number of lacO sequences to be inserted to get the best repression. Operators at the three locations were enough to successfully repress Dnmt1 promoter in the presence of lac repressor in the transient transfection reporter assay. In addition, the symmetric operator and double-operator sequences were applied to the three locations (−182, −59, and +99) in all possible combinations (FIG. 13, panel B).

5.3 Insertion of Multiple Copies of the lacO Sequence into the Endogenous Dnmt1 Promoter Using Gene Targeting.

Insertion of Multiple copies of the lacO sequence are inserted into the endogenous Dnmt1 promoter using gene targeting. Applicants obtained approximately 22 kb of genomic DNA subcloned from a phage library derived from 129/SvJae genomic DNA, stretching from about 4 kb upstream of Exon1s to 18 kb downstream of the transcription start site. A gene targeting vector was constructed using these isogenic genomic fragments to ensure efficient gene targeting (23). In certain aspects, the gene targeting is perform on wild-type J1 ES cells. In certain aspects, the lacO sequence is introduced into the endogenous Dnmt1 promoter, using a replacement-type gene-targeting vector. After homologous recombination, correctly targeted alleles contain selectable markers. In particular aspects, this selectable marker is removed by Cre recombinase, leaving one loxP site in intron 1 about 700 bp downstream from exon1s. In principle, at least three possible consequences can result from having this loxP in the intron 1. First, it can break a cis-element located at the site where the loxP is inserted. Second, it can have an effect on promoter activity by changing the distance between promoter and cis-elements located downstream of the loxP site. Third, it can interfere with splicing. Although the second and third possibilities can not be ruled out, they are less likely to be a problem since the effect of 34 by increase in distance on the activity of cis-elements located more than 700 by downstream from the promoter is unlikely to be substantial, and it is also less likely that a cis-element involved in splicing would be located at more than 500 by downstream of splicing donor site and at more than 9 kb upstream of splicing acceptor site. A Web-based putative transcription factor search was performed with the sequence around the loxP site to check the first possibility. No putative transcription factor binding sites were found with a score higher than ninety in the sequence. Low CpG density in intron 1 reduces the possibility of the presence of a strong enhancer element around the loxP site. An alternative way that does not leave any exogenous sequence besides lacO sequences in the targeted allele is to use a hit-and-run strategy, using an insertion-type gene-targeting vector (24). This strategy requires two round of homologous recombination, which extends ES cell culture, hence making it more difficult to keep ES cell undifferentiated.

5.4 Analysis of the effects of the targeted lacO insertion on endogenous Dnmt1 in the presence and absence of lac repressor in cell culture.

Analysis of the effects of the targeted lacO insertion in the presence and absence of lac repressor in cell culture. Upon successfully introducing lacO sequences into one allele of the endogenous Dnmt1 using gene targeting, a lacI gene is stably introduced into this heterozygously targeted ES cells, and a quantitative real-time RT-PCR is performed to measure the expression level of Dnmt1 in this one allele targeted ES cells. A quantitative real-time RT-PCR is specifically designed to differentiate the targeted allele from the wild type allele by taking advantage of the fact that the targeted allele has two lacO sequences in 5′UTR region. Preferably, the expression level targeted to achieve from the targeted allele is 10% of that of wild type allele. In additional aspects, the allele is introduced into the mouse germline, to generate lacI transgenics. A couple of modifications have been made in lacI gene to enhance its repressor function (described in experimental design and Example 6).

### Example 6

### Modification of the Lac Repressor

The degree of transcriptional repression by operator/repressor based prokaryotic binary systems is dependant on multiple factors, including the binding affinity between the operator and the repressor, the amount of repressor present in the biological system, the strength of the transcription unit against which the binary repression system competes, and the position of the operator (Scrable, 2002). Of these factors, binding affinity and the amount of repressor are the factors which determine the repression capacity of the system. Therefore, enhancing binding affinity and increasing the quantity of repressor would increase the reliability and general applicability of a binary system. Applicants have modified the lacI gene to 1) stabilize the Lac repressor, which results in increasing the quantity of the repressor and 2) increase the Lac repressors affinity for the Lac operator.

6.1 Quantitative improvement of the lac system by stabilizing amino acid insertion
6.2 Engineering lacI gene with tight binding mutation
6.3 Generation of lac activator by fusing lac repressor with VP16

### 6.1 Quantitative Improvement of the lac System by Stabilizing Amino Acid Insertion

The Applicants modified the repressor to include a stabilizing amino acid insertion based on the N-end rule (Varshaysky, 1996) to increase the repressor protein quantity by increasing the half-life of the protein. The N-terminal amino acid sequence of lacI protein is lysine, which is known to destabilize proteins by being a target site for ubiquitination when located at the N-terminus of a protein. Among exemplary stabilizing amino acids, three amino acids were initially selected based on the following two criteria: 1) amino acid encoded by a codon with high codon usage and 2) amino acid encoded by a codon starting with guanine. The first criterion is for better translation efficiency, and the second criterion is to increase the rate of translation initiation by completing the consensus eukaryotic translation initiation sequence. Glycine and valine were chosen because these two amino acids meet both criteria. Alanine was also chosen since it meets both criteria, although it is not a stabilizing amino acid in mammal.

Applicants introduced a glycine, a valine, or an alanine, a known stabilizing amino acid, to the N-terminus of lacIY before the lysine (lacIAY and IacIGY). This insertion not only adds a stabilizing amino acid to the repressor, but also completes the Kozak sequence, the consensus eukaryotic translation initiation sequence (GCCACCATGG) (SEQ ID NO:4). In wild type lacI, the guanine after the ATG, which is known to play an important role in translation initiation (Kozak, 1997), is missing because next codon encoding lysine starts with an adenine (AAA). Applicants restored this critical guanine residue by insertion of a glycine (GGC) or alanine (GCC). Completion of the Kozak sequence could potentially increase the quantity of repressor protein through improved translation efficiency.

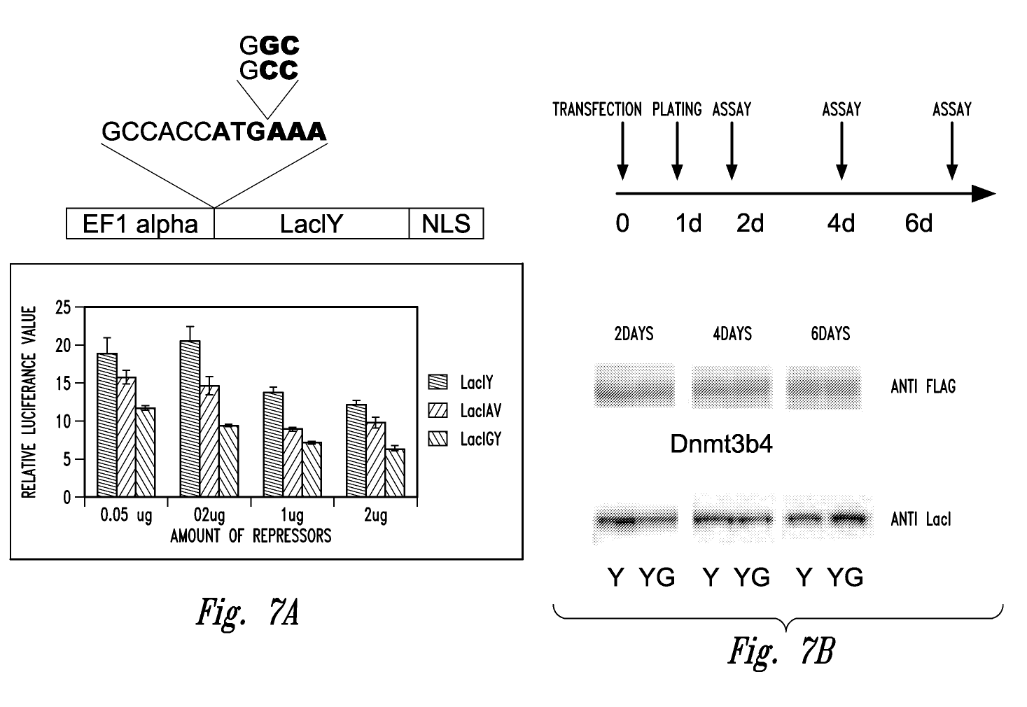

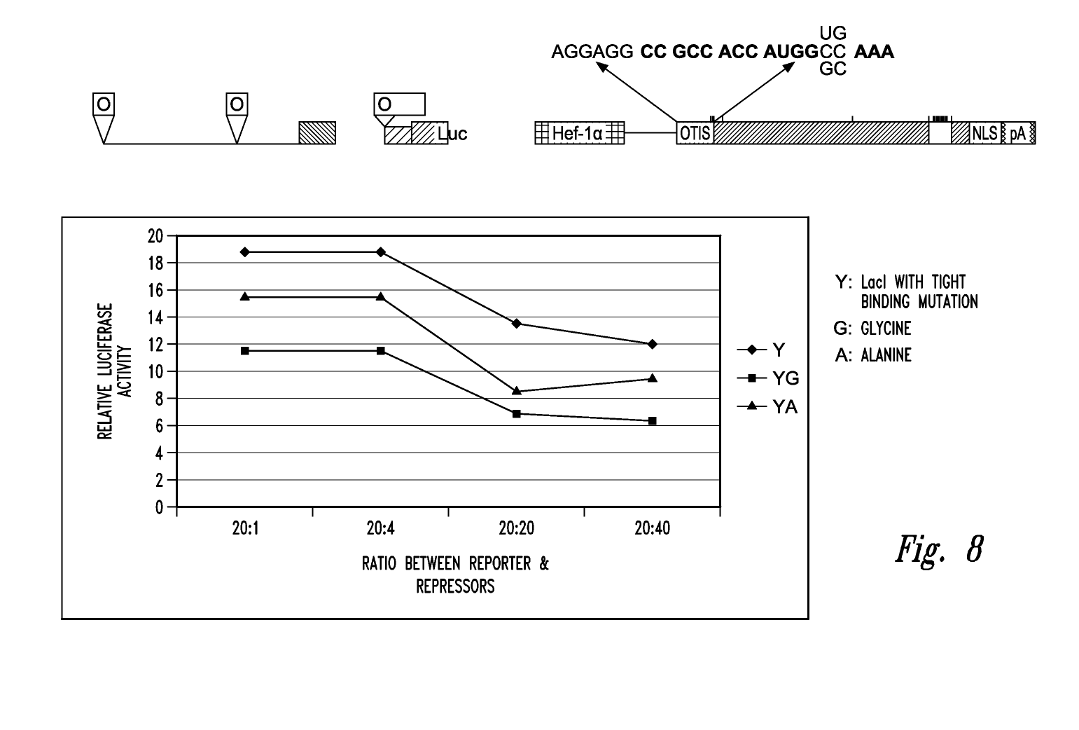

To test whether the insertion of the stabilizing amino acid indeed increased the amount of repressor and more importantly resulted in an improved repression capacity, the Applicants compared the repression capabilities and stabilities of lac repressors with and without the stabilizing amino acid. To compare the repressors for their repression capabilities, the Applicants performed reporter assays with different conditions. A fixed amount (1 mg) of a reporter plasmid was co-transfected with increased amounts of each repressor (50 ng, 200 ng, 1 mg, and 2 mg) (FIG. 7, panel A). Both lacIAY and lacIGY repressed the CMV promoter substantially better than the repressors without the stabilizing amino acid at all four conditions, and lacIGY showed the best repression (FIG. 7, panel A). This result indicates that the insertion of the stabilizing amino acid increased the repression capacity of the lac repressor. To test the effect of the modification on the stability of lac repressor, we co-transfected the lac repressor containing plasmids with a plasmid carrying Flag-tagged Dnmt3b4, which serves as a normalizing control, and measured protein quantity of each repressor at different time points after the transfection by western blot. Although the same amount of each plasmid was transfected, more repressor protein was detected for lacIGY than lacIY over time while the Dnmt3b4 protein remained constant (FIG. 7, panel B). This suggests that the insertion of the glycine resulted in increased protein levels through increased stability of the protein rather than improved translational efficiency.

Results. Lac repressor modified with those three amino acids were constructed, and tested in reporter assay system. All lac repressor with this modification showed increased repression ability. The best result was observed with lac repressor with glycine addition (˜2 times better than wild type repressor).

6.2 Engineering lacI Gene with Tight Binding Mutation

Two tight binding mutations (P3Y and S61 L) have been introduced into lacI gene, and showed tighter binding affinity to its operator sequence in Ecoli (25). In particular aspects, these tight binding mutants are introduced into mammalian cells (e.g., to provide enhanced repression ability in luciferase reporter assay system in NIH/3T3 cell line).

Results. Three lac repressors with tight binding mutation (P3Y, S61 L, and P3Y&S61 L) have been constructed and tested these mutants in the Applicants' reporter assay system with luciferase gene driven by Dnmt1 promoter modified with lacO sequences in NIH/3T3 cell line. Of the three tight binding mutants, P3Y mutants showed the best repression ability, ˜5 times better than wild type repressor similar to the results seen in Ecoli (25).

6.3 Generation of lac Activator by Fusing lac Repressor with VP16

Generation of lac activator by fusing lac repressor with VP16. With access to the Dnmt1 promoter through lac operator/repressor interaction, this interaction is, according to particular aspects used to overexpress Dnmt1 using lac repressor fused with transcriptional activator, VP16. Lac repressor/VP16 fusion protein has been successfully used in mammalian cells (27). With overexpression of Dnmt1 by lac repressor/VP16, the inventive binary transcriptional regulation system allows to not only down-regulate, but also up-regulate endogenous Dnmt1 expression in vivo. Additional aspects provide various Dnmt1 promoters with lacO at different locations to provide optimal locations for activation using luciferase reporter assay.

VP16 was introduced to lacI gene at three locations, one of which is the same location described in (27). Two other locations were chosen based on the knowledge of functional domains of lac repressor and restriction sites availability. Both tight binding mutant P3Y and wild type lac repressor were engineered. Tight binding mutants with VP16 fusion showed better activation ability than wild type lac repressor/VP16. Dnmt1 promoter was overexpressed up to 2.5-fold with one of tight binding mutant/VP16 constructs (LAVPN) in transient transfection reporter assay in NIH/3T3 cells. By introducing multiple VP16s (3 and 4 in a row), Dnmt1 was overexpressed up to 4-fold.

### Example 7

### Reversible Repression of the Dnmt1 Promoter by the lac Repressor and IPTG was Demonstrated

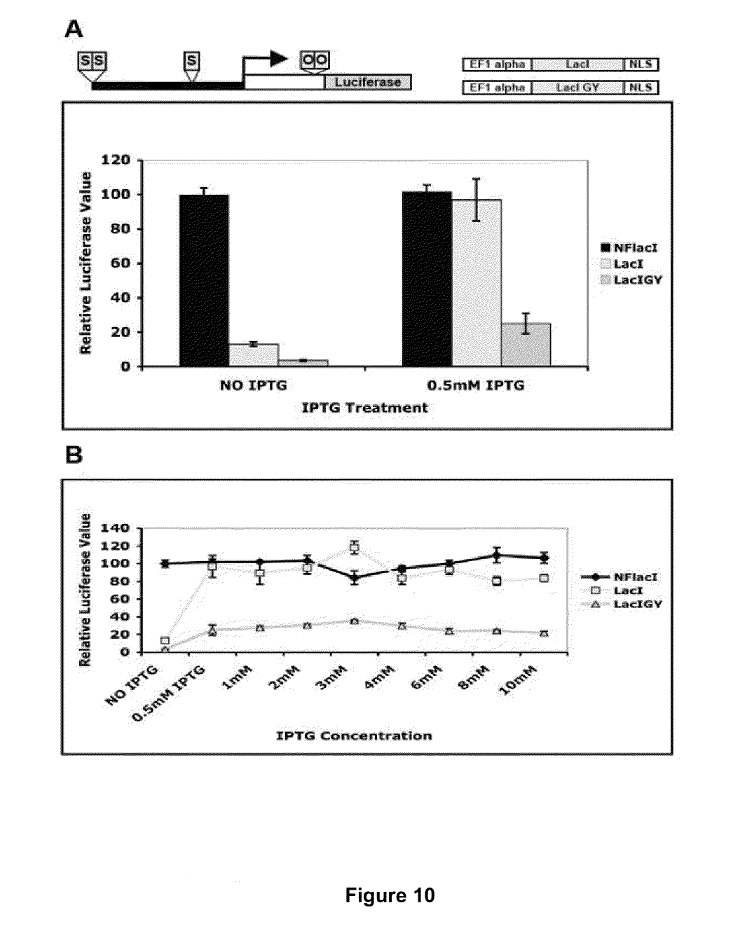

Applicants tested whether the lac repressor can repress the modified Dnmt1 promoter. A reporter plasmid with the modified Dnmt1 promoter and the lacI or lacIGY plasmids were transiently introduced into NIH3T3 cells in a 1:1 molar ratio. Both lac repressors successfully repressed the operator inserted Dnmt1 promoter (FIG. 10, panel A). Almost complete repression (3.7% residual expression) was achieved with lacIGY. The repression by lacI was completely reversed by 0.5 mM IPTG treatment while only 25% restoration was achieved for lacIGY with the same IPTG treatment. To test whether the repression by lacIGY can be completely reversed by higher amounts of IPTG, increased molar concentrations of IPTG (0.5 mM to 10 mM) were applied (FIG. 10, panel B). The best restoration levels of transcriptional expression was at 3 mM IPTG, which was 40% of unregulated expression.

### Example 8

### General Applicability of the lac System was Demonstrated

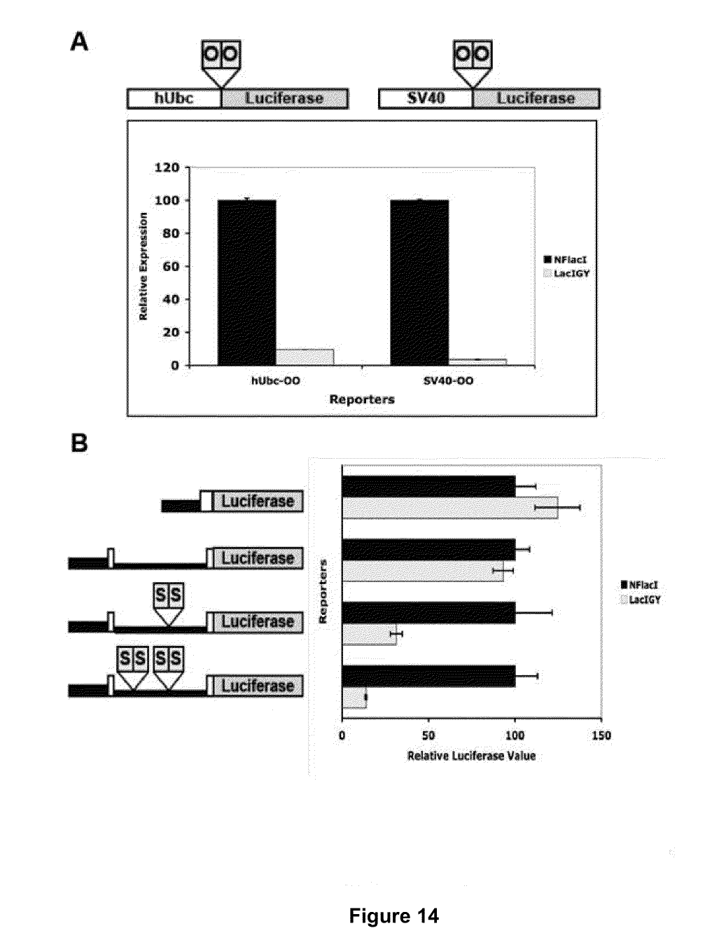

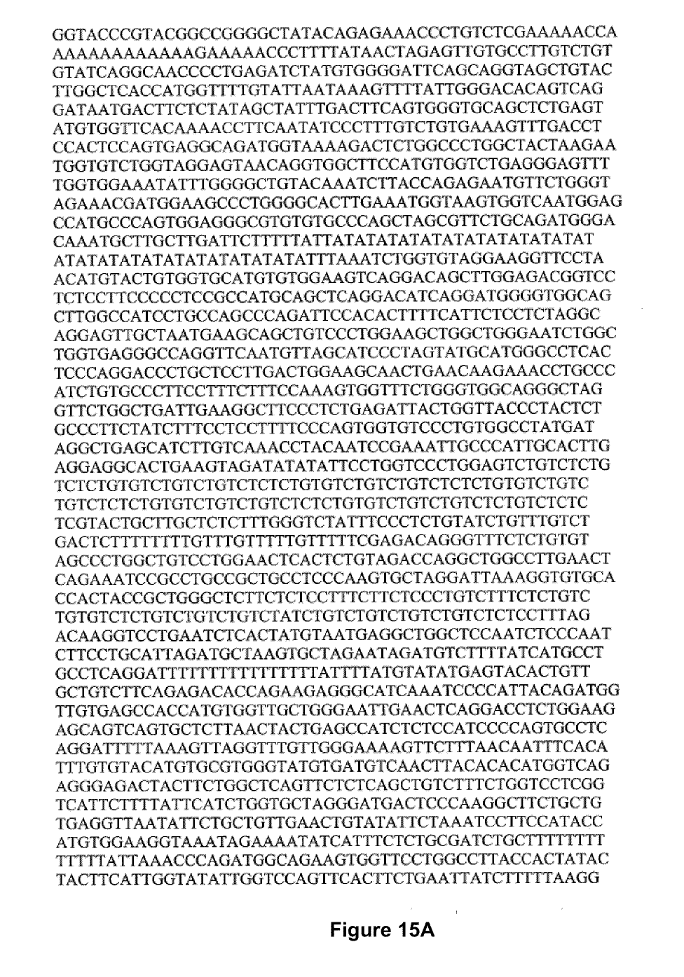

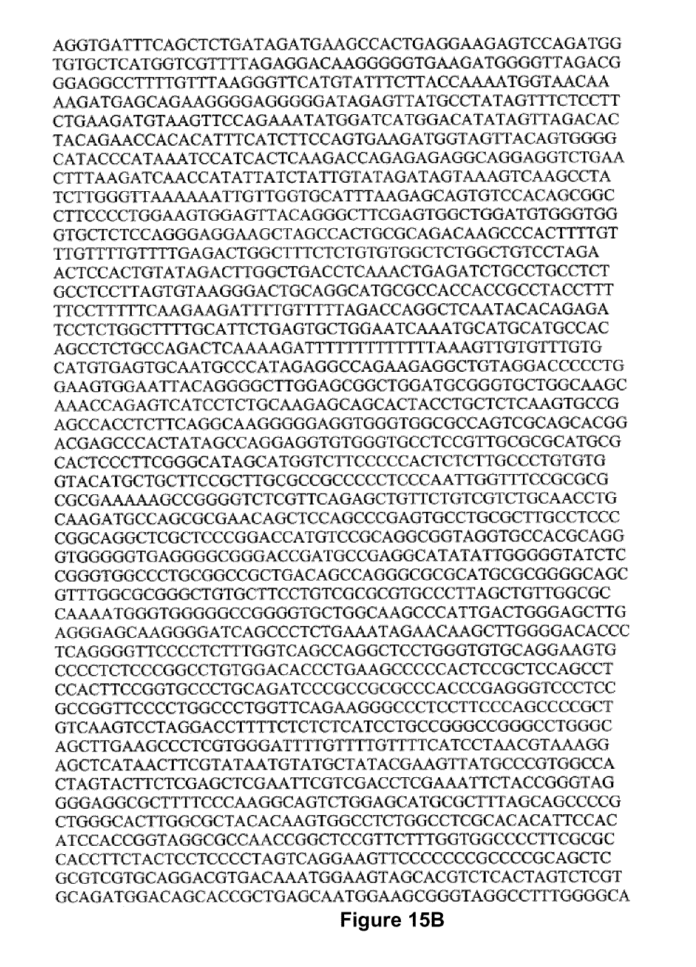

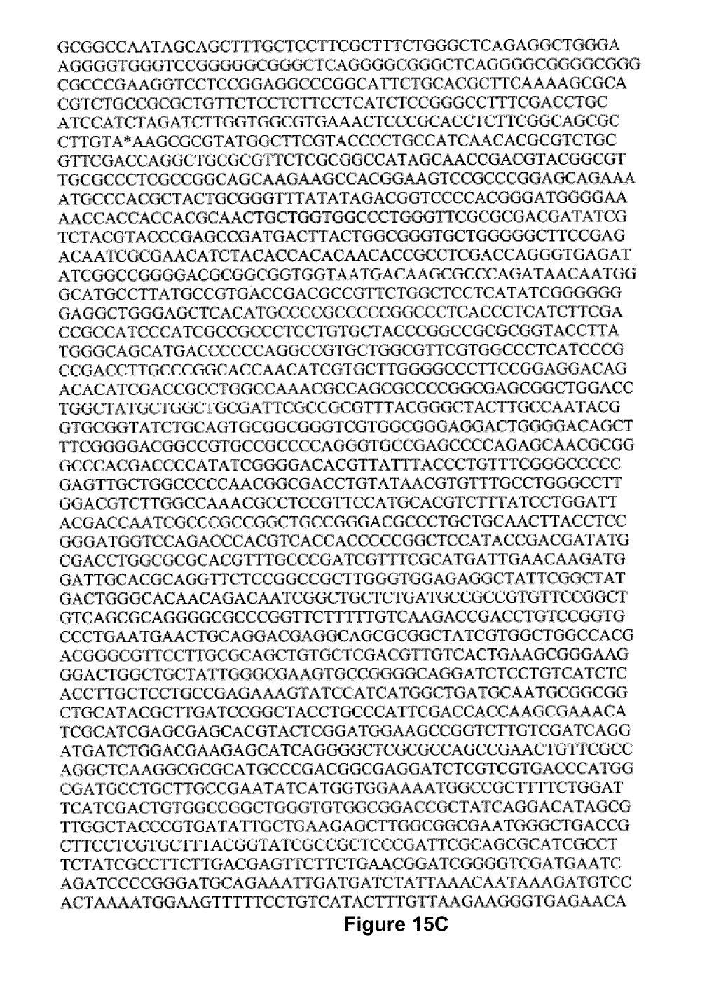

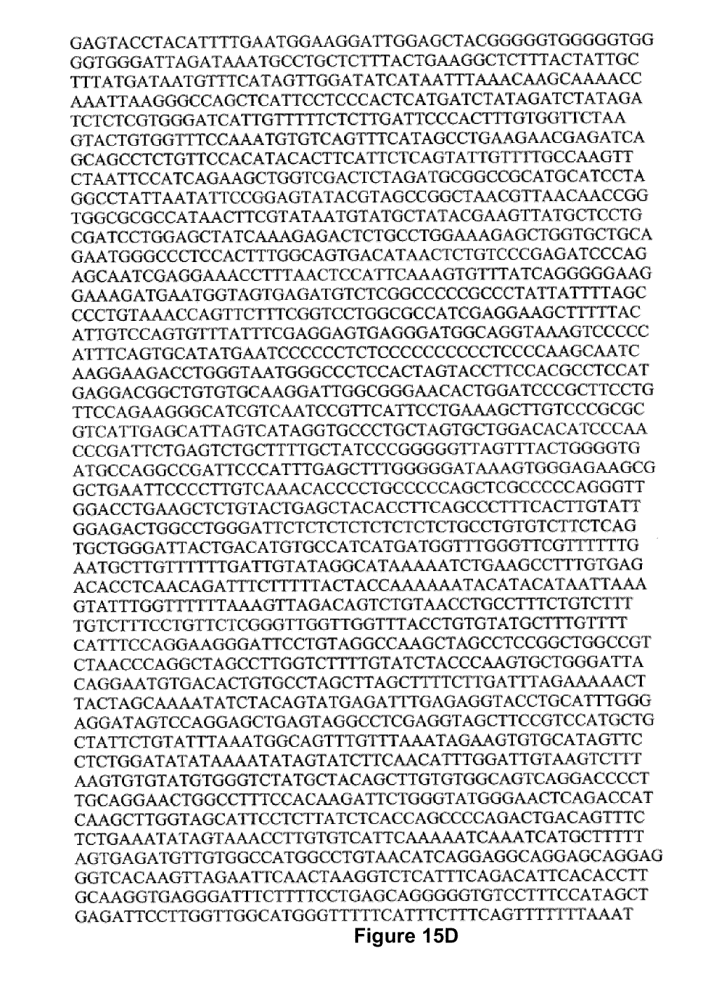

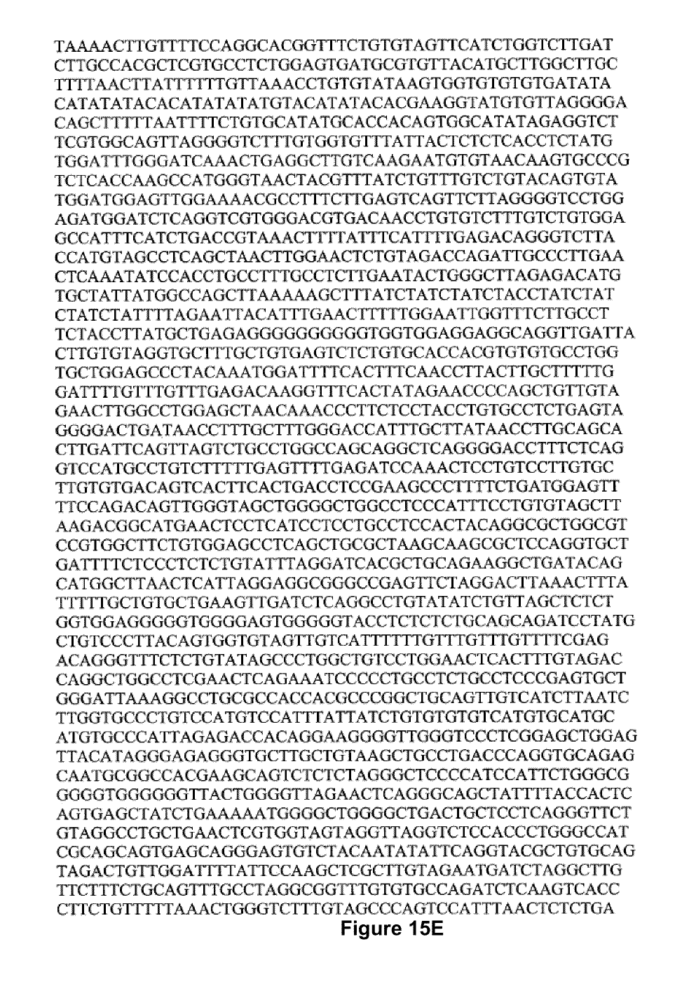

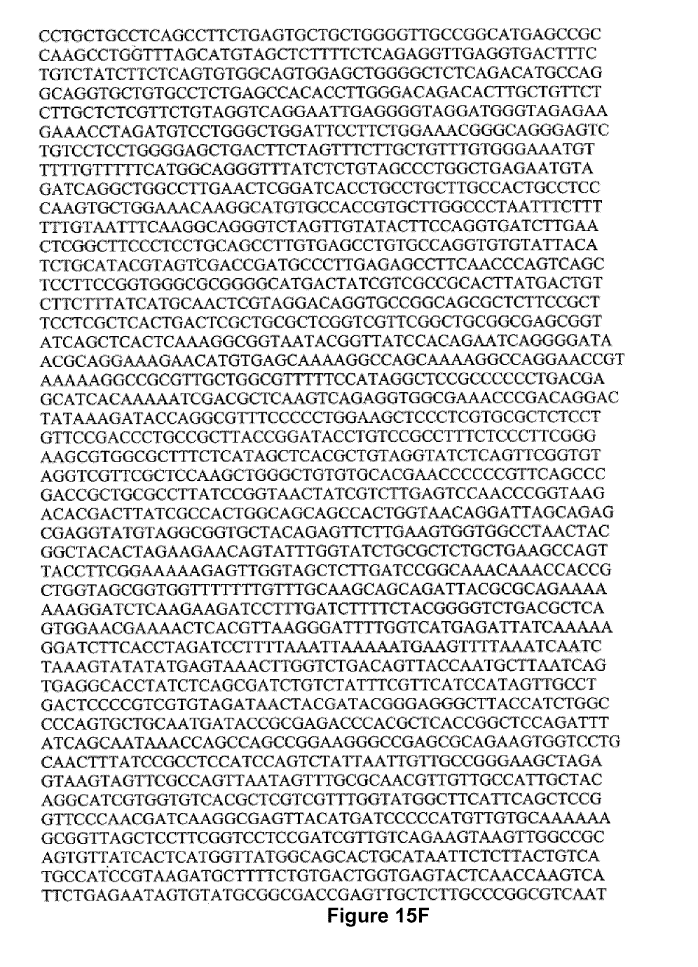

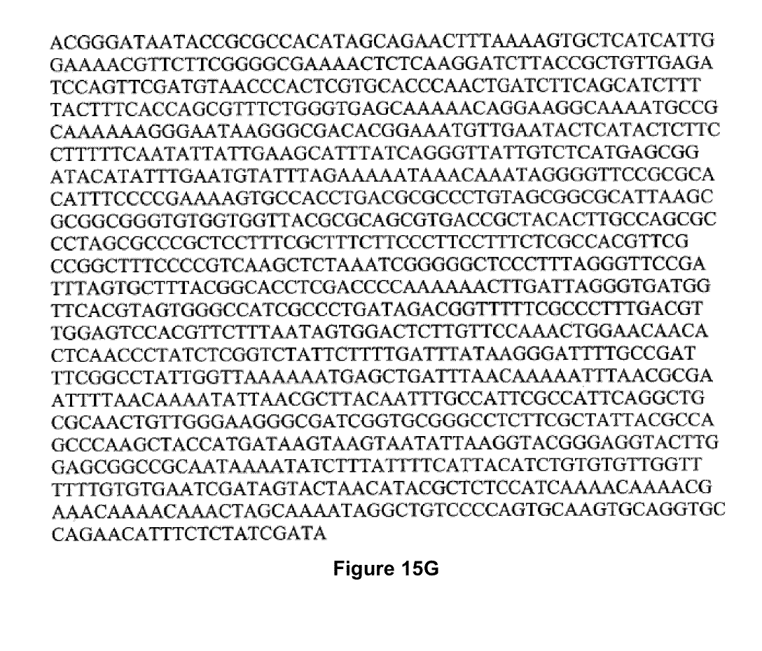

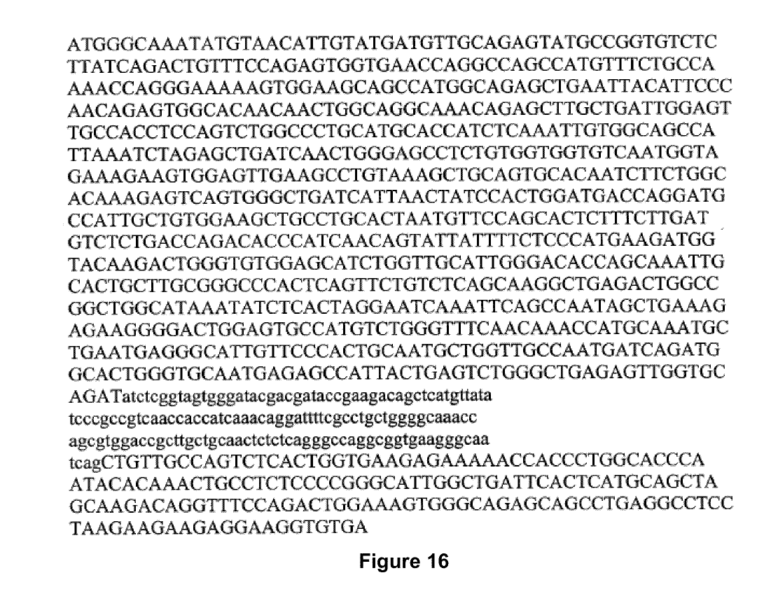

To test the general applicability of the repression principle Applicants applied the lac system to other promoters. Applicants modified the simian virus 40 (SV40) and the human ubiquitin C (hUbc) promoters with lac operators and subjected them to a reporter assay. As seen with the CMV and the Dnmt1 promoters, these promoters were almost completely repressed by the modified lac repressor (FIG. 14, panel A). According to certain embodiments, this result indicates that the modifications the Applicants introduced to the lac operator/repressor system have substantially improved reliability and general applicability of the system and suggests that our repression approach can be successfully applied to other eukaryotic promoters.

### Example 9

### Blocking Transcription Elongation by lac Repressor was Demonstrated

According to certain embodiments, the best placement of the lac operator is within intronic regions. Reasons for this example include: 1) a significant portion of eukaryotic genes has more than one promoter, 2) insertion locations identified with a certain cell type may not be optimal for other cell types, and 3) some genes may not be able to accommodate operator sequences at their 5′UTR, the most crucial location for the repression. In certain embodiments the issues listed above may be addressed by inserting the lac operator sequences into intronic regions if the repressor is able to impede transcription elongation. In certain embodiments, the modifications that Applicants integrated into the lac operator/repressor system might allow the blockage of transcription elongation through the enhanced operator-repressor interaction. To test this, the rabbit β-globin intron was modified with the symmetric double operators under the control of Dnmt1 promoter. In a transient reporter assay, expression from the Dnmt1 promoter was significantly repressed, indicating that the modified lac repressor (lacIGY) was able to block the transcription elongation (FIG. 14, panel B). According to certain embodiments, the relatively close location of the lac operators to the promoter (448 by downstream from the transcription start site) allowed the lac repressors to interfere with transcription initiation.

### Example 10

### A Conditional Regulatory System of Dnmt1 Gene Expression in the Mouse is Developed

Introduction of the Dnmt1-lacO allele into the mouse germline. In particular aspects, the targeted allele is introduced into the mouse germline. In certain embodiments, the targeted allele is maintained in 129/svJae mice, and is optionally also backcrossed to C57BL/6 for testing with lacI transgenics.

Generation of lacI transgenic mice and testing of the binary system. In certain aspects, two lines of transgenic mice are generated, one with ubiquitous lacI expression and another with tissue specific lacI expression to test our binary system at the mouse and a tissue level. (28) For the former, three promoters, ubiquitine, EF1α, and human β-actin promoters are first compared. The strongest promoter to have a robust ubiquitous lac repressor or activator expression is chosen. For the latter, the FabpI^{4x at-132} modified fatty acid binding promoter (28) is preferably used, which is well-characterized and provides excellent tissue-specific expression in the colonic crypt epithelium and small intestine, with some additional expression in the bladder (29). Quantitative RT-PCR is performed to confirm that this binary system works in Dnmt1-lacO mice carrying the lacI transgene, compared to non-transgenic sibling Dnmt1-lacO controls. In particular aspects of this binary transcriptional repression/activation system transcriptional repression/activation can be reversed by IPTG (17). Transgenic mice are first generated expressing lac repressor/activator with tight binding mutation and stabilizing amino acid. Alternatively, lacI gene without these modifications can be used. In additional aspects, the system is expanded to include tissue-specific expression in other tissue types for modulating and determining the effects of modulated Dnmt1 expression on tumor model systems.

### Example 11

### A Conditional Regulatory System of Dnmt1 Gene Expression in the Mouse was Demonstrated

This example describes the in vivo demonstration of the repression system. After the establishment of the binary systems in vitro, Applicants tested the system in mice. Applicants introduced the lac operators to the endogenous Dnmt1 promoter through gene-targeting, and generated transgenic lines expressing the lac repressor. In the mouse experiments, Applicants found that the improved lac system successfully repressed endogenous Dnmt1 expression tissue-specifically and ubiquitously. The repression was potent enough to reproduce the embryonic lethal phenotype of genetic knock-outs when applied ubiquitously. Importantly, this lethal phenotype was rescued by treating the pregnant mice with IPTG.

Due to the essential requirement of Dnmt1 for development and viability, in vivo characterization of Dnmt1 functions through knock-out approaches have been very limited (Li et al., 1992; Jackson-Grusby et al., 2001; Lei et al., 1996; Holm et al., 2005). Now, with this novel approach, Applicants circumvented the lethality problem and successfully produced Dnmt1 knockdown mouse models for the first time, which could serve as a useful resource for studying the role of DNA methylation in many biological processes and diseases.

### Example 12

### Induced Overexpression of Endogenous Dnmt1 was Demonstrated

Applicants applied the binary activation system to the endogenous Dnmt1 to test the novel gene activation principle on an endogenous gene. To induce overexpression of Dnmt1 in mice, Applicants generated Dnmt1LGT mice that contain tet operators in the endogenous Dnmt1 promoter in addition to the lac operaor sequences. Mice were then crossed with existing tetVP16 or rtetVP16 transgenic lines. No successful transgenic mouse models have been reported for Dnmt1 upregulation. However, the approach enabled production of mouse models exhibiting elevated Dnmt1 expression in various tissues. These mouse models provide a new opportunity for the functional analysis of Dnmt1 and facilitate the gain-of-function genetics for Dnmt1.

### Example 13

### The Role of DNA Methylation in Cancer Using Binary Transcriptional Regulatory System in Mouse Models

With the desired level of Dnmt1 expression (repression and activation), which does not cause non-viability of either cells or mice, the role of DNA methylation in cancer is investigated with this system in following three mouse experiments:
1) Downregulation of Dnmt1 in mice predisposed to develop cancer
2) Overexpression of Dnmt1 in normal mice and in the mice predisposed to develop cancer
3) Downregulation of Dnmt1 in the mice predisposed to develop cancer only after tumor formation.
For the first experiment, a Dnmt1-lacO mouse carrying lacI transgene is crossed with Apc^{Min/+} mice and Mlh^{-/-} mice. With Dnmt1 heterozygous and hypomorphic mice (Dnmt1^{-/R}), we have previously shown that reduced expression of Dnmt1 results in the suppression of benign neoplasia in the intestines of Apc^{Min/+} mice and of benign and malignant intestinal tumors in Mlh^{-/-} mice (30, 31). In particular aspects, Dnmt1 expression is repressed as low as that of Dnmt1^{-/R} mice. The possible effects of lacI transgene on tumorigenesis are controlled by crossing lacI transgenic mice with Apc^{Min/+} mice and Mlh^{-/-} mice. The difference in the underlying mechanisms for the reduced expression of Dmnt1 between Dnmt1^{-/R} and Dnmt1-lacO mouse carrying lad transgene may result in different phenotypes. The repression mechanism of Dnmt1^{-/R} mice does not involve trans-acting factors, but that of Dnmt1-lacO mouse carrying lacI transgene involves trans-acting factor, lac repressor. It is possible but not necessary that reduced expression level of Dnmt1 in Dnmt1-lacO mouse carrying lacI transgene might be not as stable as that of Dnmt1^{-/R} over the time of mouse development. For the second experiment, a Dnmt1-lacO mouse carrying lac/VP16 transgene is crossed with normal mice, Apc^{Min/+} mice and Mlh^{-/-} mice to confirm that Dnmt1 overexpression enhances tumorigenesis. Where no phenotypic changes are observed, several interpretations are possible. First, together with the results from the experiment with Dnmt1^{-/R} and Apc^{Min/+} mice it suggests that sufficient Dnmt1 expression level is required for and involved in tumorigenesis, but Dnmt1 dose not play a causal role. Second, Dnmt1 may have a causal effect, but Overexpression of Dnmt1 alone is not sufficient to enhance tumorigenesis. Third Dnmt1 may play a causal role in tumorigenesis, but not through its overexpression. For third experiment, the same cross is performed as for the first experiment. But, in this experiment, lac repressor is blocked by IPTG. It has been shown that IPTG could act transplacentally in the mouse (17), so if the mother is treated with IPTG (10 mM IPTG in drinking water), lac repressor can be blocked in the offspring, resulting normal level of Dnmt1 expression. IPTG treatment will be stopped after the offspring develop tumor to confirm the role of DNA methylation in tumor progression and maintenance. Apc^{Min/+} mice and Mlh^{-/-} mice treated with IPTG are included in certain aspects of this experiment as a control for the effect of IPTG on tumorigenesis.

According to certain aspects, despite the fact that controlling endogenous gene expression by a binary transcriptional regulatory system is a relatively new application, recent work has been encouraging (17, 32), and the present engineered lac repressor provides a better repression capacity. Additional aspects comprise generating hybrid repressors with a mammalian repressor, for example, the KRAB domain of Human KoxI, which has been successfully applied to Tet repressor (33). Additional aspects provide efficient expression of lacI gene. Additional aspects provide vectors comprising reduced numbers of CpG dinucleotides to reduce DNA methylation-mediated silencing in transgenic mice (see, e.g., 17, 32).

### REFERENCES CITED

1. Robertson, K. D. 2002. DNA methylation and chromatin—unraveling the tangled web. Oncogene 21, 5361-5379.
2. Laird, P. W. 2003. The power and the promise of DNA methylation markers. Nat Rev Cancer 3, 253-266.
3. Bird, A. 2002. DNA methylation patterns and epigenetic memory. Genes Dev 16, 6-21.
4. Baylin, S. B., and Herman, J. G. 2000. DNA hypermethylation in tumorigenesis: epigenetics joins genetics. Trends Genet. 16, 168-174.
5. Jones, P. A., and Baylin, S. B. 2002. The fundamental role of epigenetic events in cancer. Nat Rev Genet. 3, 415-428.
6. Lewandoski, M. 2001. Conditional control of gene expression in the mouse. Nat Rev Genet. 2:743-755.
7. Jackson-Grusby, L., C. Beard, R. Possemato, M. Tudor, D. Fambrough, G. Csankovszki, J. Dausman, P. Lee, C. Wilson, E. Lander, and R. Jaenisch. 2001. Loss of genomic methylation causes p53-dependent apoptosis and epigenetic deregulation. Nat Genet. 27:31-39.
8. Lee, P. P., D. R. Fitzpatrick, C. Beard, H. K. Jessup, S. Lehar, K. W. Makar, M. Perez-Melgosa, M. T. Sweetser, M. S. Schlissel, S. Nguyen, S. R. Cherry, J. H. Tsai, S. M. Tucker, W. M. Weaver, A. Kelso, R. Jaenisch, and C. B. Wilson. 2001. A critical role for Dnmt1 and DNA methylation in T cell development, function, and survival. Immunity 15:763-774.
9. Gossen, M., A. L. Bonin, S. Freundlieb, and H. Bujard. 1994. Inducible gene expression systems for higher eukaryotic cells. Curr Opin Biotechnol 5:516-520.
10. Gossen, M., S. Freundlieb, G. Bender, G. Muller, W. Hillen, and H. Bujard. 1995. Transcriptional activation by tetracyclines in mammalian cells. Science 268:1766-1769.
11. Manickan, E., J. Satoi, T. C. Wang, and T. J. Liang. 2001. Conditional liver-specific expression of simian virus 40 T antigen leads to regulatable development of hepatic neoplasm in transgenic mice. J Biol Chem 276:13989-13994.
12. Kramer, H., Niemoller, M., Amouyal, M., Revet, B., von Wilcken-Bergmann, B., and Muller-Hill, B. 1987. lac repressor forms loops with linear DNA carrying two suitably spaced lac operators. Embo J 6, 1481-1491.
13. Albert, B., Johnson, A., Lewis, J., Raff, M., Roberts, K, and P. Walter. Molecular Biology of THE CELL. Forth edition, 395-399.
14. Brown, M., J. Figge, U. Hansen, C. Wright, K. T. Jeang, G. Khoury, D. M. Livingston, and T. M. Roberts. 1987. lac repressor can regulate expression from a hybrid SV40 early promoter containing a lac operator in animal cells. Cell 49:603-612.
15. Deuschle, U., W. K. Meyer, and H. J. Thiesen. 1995. Tetracycline-reversible silencing of eukaryotic promoters. Mol Cell Biol 15:1907-1914.
16. Edamatsu, H., Y. Kaziro, and H. Itoh. 1997. Inducible high-level expression vector for mammalian cells, pEF-LAC carrying human elongation factor 1alpha promoter and lac operator. Gene 187:289-294.
17. Cronin, C. A., W. Gluba, and H. Scrable. 2001. The lac operator-repressor system is functional in the mouse. Genes Dev 15:1506-1517.
18. Deuschle, U., R. A. Hipskind, and H. Bujard. 1990. RNA polymerase II transcription blocked by Escherichia coli lac repressor. Science 248:480-3.
19. Mertineit, C., J. A. Yoder, T. Taketo, D. W. Laird, J. M. Trasler, and T. H. Bestor. 1998. Sex-specific exons control DNA methyltransferase in mammalian germ cells. Development 125:889-897.
20. Howell, C. Y., T. H. Bestor, F. Ding, K. E. Latham, C. Mertineit, J. M. Trasler, and J. R. Chaillet. 2001. Genomic imprinting disrupted by a maternal effect mutation in the dnmt1 gene. Cell 104:829-838.
21. Kishikawa, S., Murata, T., Kimura, H., Shiota, K., and Yokoyama, K. K. 2002. Regulation of transcription of the Dnmt1 gene by Sp1 and Sp3 zinc finger proteins. Eur J Biochem 269, 2961-2970.
22. Kimura, H., Nakamura, T., Ogawa, T., Tanaka, S., and Shiota, K. 2003. Transcription of mouse DNA methyltransferase 1 (Dnmt1) is regulated by both E2F-Rb-HDAC-dependent and -independent pathways. Nucleic Acids Res 31, 3101-3113.
23. to Riele, H., E. C. Robanus Maandag, and A. Berns. 1992. Highly efficient gene targeting in embryonic stem cells through homologous recombination with isogenic DNA constructs. Proc Natl Acad Sci USA 89:5128-32.
24. Hasty, P., R. Ramirez-Solis, R. Krumlauf, and A. Bradley. 1991. Introduction of a subtle mutation into the Hox-2.6 locus in embryonic stem cells [published erratum appears in Nature 1991 Sep. 5; 353(6339):94]. Nature 350:243-6.
25. Kolkhof, P. 1992. Specificities of three tight-binding Lac repressors. Nucleic Acids Res 20:5035-5039.
26. Varshaysky, A. 1996. The N-end rule: functions, mysteries, uses. Proc Natl Acad Sci USA 93, 12142-12149.
27. Labow, M. A., Baim, S. B., Shenk, T., and Levine, A. J. 1990. Conversion of the lac repressor into an allosterically regulated transcriptional activator for mammalian cells. Mol Cell Biol 10, 3343-3356.
28. Saam, J. R., and J. I. Gordon. 1999. Inducible gene knockouts in the small intestinal and colonic epithelium. J Biol Chem 274:38071-38082.
29. Wong, M. H., J. R. Saam, T. S. Stappenbeck, C. H. Rexer, and J. I. Gordon. 2000. Genetic mosaic analysis based on Cre recombinase and navigated laser capture microdissection. Proc Natl Acad Sci USA 97:12601-12606.
30. Eads, C. A., A. E. Nickel, and P. W. Laird. 2002. Complete genetic suppression of polyp formation and reduction of CpG-island hypermethylation in ApcMin/+Dnmt1-hypomorphic mice. Cancer Res 62:In Press.
31. Trinh, B. N., T. I. Long, A. E. Nickel, D. Shibata, and P. W. Laird. 2002. DNA methyltransferase deficiency modifies cancer susceptibility in mice lacking DNA mismatch repair. Mol Cell Biol 22:In Press.
32. Scrable, H., and P. J. Stambrook. 1997. Activation of the lac repressor in the transgenic mouse. Genetics 147:297-304.
33. Deuschle, U., W. K. Meyer, and H. J. Thiesen. 1995. Tetracycline-reversible silencing of eukaryotic promoters. Mol Cell Biol 15:1907-1914.

1. A method for transcriptional control of an endogenous gene, comprising:
introducing, into a suitable region of a target endogenous mammalian target gene sequence of a mammalian cell having a genome, at least one cis element of at least one exogenous binary regulatory DNA sequence;
introducing, into the genome of the mammalian cell, an expression cassette/vector encoding at least one corresponding trans element of the at least one binary regulatory DNA sequence, wherein the at least one cis element is operative with the target gene sequence and the at least one trans element to provide for transcriptional control of the endogenous target gene expression by the at least one binary regulatory DNA sequence.

2. The method of claim 1, wherein introducing of the at least one cis element comprises introducing multiple cis elements.

3. The method of claim 1, wherein introducing of the at least one trans element comprises introducing multiple trans elements.

4. The method of claim 1, wherein the expression cassette/vector encoding the at least one corresponding trans element of the binary regulatory DNA sequence comprises mammalian promoter and/or regulatory sequences.

5. The method of claim 1, further comprising administration of an agent suitable to modulate the intracellular interaction between the cis and trans elements of the binary regulatory DNA sequences.

6. The method of claim 5, wherein the agent is at least one selected from the group consisting of allolactose, lactose, IPTG, tetracyclines, and galactose.

7. The method of claim 1, wherein the cis and trans exogenous binary regulatory DNA sequences are of heterologous origin.

8. The method of claim 7, wherein the cis and trans exogenous binary regulatory DNA sequences those of microorganisms, including bacteria and yeast.

9. The method of claim 1, wherein the binary regulatory DNA sequences are selected from the group consisting of lac operator/repressor, tet operator/repressor, Gal4 operator/repressor, and functional variants (muteins, fusions, deletions, insertions, fragments, derivatives, etc) thereof.

10. The method of claim 1, wherein transcriptional control of the endogenous gene expression by the binary regulatory DNA sequences comprises transcriptional repression or transcriptional activation.

11. The method of claim 1, wherein introducing at least one cis element of at least one exogenous binary regulatory DNA sequence comprises recombination.

12. The method of claim 1, wherein the target gene comprises the Dnmt1 gene.

13. The method of any one of claims 1 through 12, comprising:
introducing the at least one cis element into at least one cell of a first transgenic mammal;
introducing the at least one trans element into at least one cell of a second transgenic mammal
crossing the first and second transgenic mammals to provide at least one cell, or provide at least one progeny animal with at least one cell, wherein the at least one cis element is operative with the target gene and the trans element to provide for transcriptional control of the endogenous gene expression in the at least one cell by the at least one binary regulatory DNA sequence.

14. A mammalian cell, comprising at least one of:
an endogenous target gene sequence having suitably inserted therein at least one cis element of at least one exogenous binary regulatory DNA sequence; and
an expression cassette/vector, within the genome of the mammalian cell, encoding at least one corresponding trans element of the at least one binary regulatory DNA sequence, wherein the at least one cis element is operative with the target gene sequence and the at least one trans element to provide for transcriptional control of the endogenous target gene expression by the at least one binary regulatory DNA sequence.

15. The cell of claim 14, wherein the cell is that of, or within a transgenic animal.

16. The cell of claim 14, wherein the cell is an embryonic stem cell.

17. The method of claim 14, wherein the target gene comprises the Dnmt1 gene.

18. A lacI repressor, comprising a stabilizing amino acid adjacent to the N-terminal lysine.

19. The lacI repressor of claim 18, wherein the stabilizing amino acid is Gly, Ala, or Val.

20. The lacI repressor of claim 19, wherein the lacI sequence comprises a consensus GCCACCATGG (SEQ ID NO:1), or GNCACCATGG (SEQ ID NO:2) sequence, wherein the N is selected from the group consisting of cytosine, guanine, and thymidine.

21. A lacI repressor, comprising at least one mutation selected from the group consisting of proline to tyrosine change at the third amino acid residue and serine to leucine change at amino acid residue 61.

22. The lacI repressor, of any claims 18 through 21, wherein the lacI repressor is further fused to another protein, wherein the another protein is VP16.

23. A lacI repressor, comprising a fusion protein, wherein the fusion protein is VP16.

24. A lac operator, comprising a symmetric lac operator sequence consisting of TGTGGAATTGTGAGC-GCTCACAATTCCACA (SEQ ID NO:3).

25. A method for bidirectional cloning, comprising:
generating at least one deletion mutant construct; and
inserting at least one regulatory sequence at different locations with respect to the at least one deletion mutant construct, wherein only three restriction enzymes are used, and wherein the generating at least one deletion mutant construct and the inserting at least one regulatory sequence are synchronized.

26. The method of claim 21, wherein the regulatory sequence is an operator.

27. The method of claim 22, wherein the operator is selected from the group consisting of the lac operator, the lac operator with at least one lacI binding consensus sequence, the lac operator with at least one lacI binding symmetrical sequence TGTGGAATTGTGAGC-GCTCACAATTCCACA (SEQ ID NO:3), the tet operator, and the Gal4 regulatory sequence.

28. The method of claim 1, further comprising modifying the number or pattern of CpG dinucleotides within the expression cassette/vector to modulate DNA methylation-mediated silencing thereof.


SEQUENCE LISTING (12 sequences)

SEQ ID NO: 1 - DNA, 10 bp, Artificial Sequence
  lacI consensus
gccaccatgg

SEQ ID NO: 2 - DNA, 10 bp, Artificial Sequence
  lacI consensus 2
gncaccatgg

SEQ ID NO: 3 - DNA, 30 bp, Artificial Sequence
  symmetric lac operator sequence
tgtggaattgtgagcgctcacaattccaca

SEQ ID NO: 4 - DNA, 10 bp, Artificial Sequence
  consensus eukaryotic translation initiation sequence
gccaccatgg

SEQ ID NO: 5 - DNA, 15 bp, Artificial Sequence
  exemplary stabilizing amino acid insertions for Lac repressor
gccaccatggccaaa

SEQ ID NO: 6 - DNA, 14 bp, Artificial Sequence
  exemplary stabilizing amino acid insertions for Lac repressor 2
gccaccatggcaaa

SEQ ID NO: 7 - DNA, 23 bp, Artificial Sequence
  stabilized lac repressor
aggaggccgccaccangggcaaa

SEQ ID NO: 8 - DNA, 23 bp, Artificial Sequence
  stabilized lac repressor
aggaggccgccaccanggccaaa

SEQ ID NO: 9 - DNA, 23 bp, Artificial Sequence
  stabilized lac repressor 3
aggaggccgccaccanggngaaa

SEQ ID NO: 10 - DNA, 31 bp, Artificial Sequence
  stabilized lac repressor 1
ngtggaattgtgagcggataacaatttcacn

SEQ ID NO: 11 - DNA, 14920 bp, Artificial Sequence
  targeting vector for Dnmt1 promoter
ggtacccgtacggccggggctatacagagaaaccctgtctcgaaaaaccaaaaaaaaaaaaagaaaaacccttttataactagagttgtgccttgtctgtgtatcaggcaacccctgagatctatgtggggattcagcaggtagctgtacttggctcaccatggttttgtattaataaagttttattgggacacagtcaggataatgacttctctatagctatttgacttcagtgggtgcagctctgagtatgtggttcacaaaaccttcaatatccctttgtctgtgaaagtttgacctccactccagtgaggcagatggtaaaagactctggccctggctactaagaatggtgtctggtaggagtaacaggtggcttccatgtggtctgagggagttttggtggaaatatttggggctgtacaaatcttaccagagaatgttctgggtagaaacgatggaagccctggggcacttgaaatggtaagtggtcaatggagccatgcccagtggagggcgtgtgtgcccagctagcgttctgcagatgggacaaatgcttgcttgattctttttattatatatatatatatatatatatatatatatatatatatatatatatatttaaatctggtgtaggaaggttcctaacatgtactgtggtgcatgtgtggaagtcaggacagcttggagacggtcctctccttccccctccgccatgcagctcaggacatcaggatggggtggcagcttggccatcctgccagcccagattccacacttttcattctcctctaggcaggagttgctaatgaagcagctgtccctggaagctggctgggaatctggctggtgagggccaggttcaatgttagcatccctagtatgcatgggcctcactcccaggaccctgctccttgactggaagcaactgaacaagaaacctgcccatctgtgcccttcctttctttccaaagtggtttctgggtggcagggctaggttctggctgattgaaggcttccctctgagattactggttaccctactctgcccttctatctttcctccttttcccagtggtgtccctgtggcctatgataggctgagcatcttgtcaaacctacaatccgaaattgcccattgcacttgaggaggcactgaagtagatatatattcctggtccctggagtctgtctctgtctctgtgtctgtctgtctctctgtgtctgtctgtctctctgtgtctgtctgtctctctgtgtctgtctgtctctctgtgtctgtctgtctctgtctctctcgtactgcttgctctctttgggtctatttccctctgtatctgtttgtctgactcttttttttgtttgtttttgtttttcgagacagggtttctctgtgtagccctggctgtcctggaactcactctgtagaccaggctggccttgaactcagaaatccgcctgccgctgcctcccaagtgctaggattaaaggtgtgcaccactaccgctgggctcttctctcctttcttctccctgtctttctctgtctgtgtctctgtctgtctgtctatctgtctgtctgtctgtctctcctttagacaaggtcctgaatctcactatgtaatgaggctggctccaatctcccaatcttcctgcattagatgctaagtgctagaatagatgtcttttatcatgcctgcctcaggattttttttttttttttattttatgtatatgagtacactgttgctgtcttcagagacaccagaagagggcatcaaatccccattacagatggttgtgagccaccatgtggttgctgggaattgaactcaggacctctggaagagcagtcagtgctcttaactactgagccatctctccatccccagtgcctcaggatttttaaagttaggtttgttgggaaaagttctttaacaatttcacatttgtgtacatgtgcgtgggtatgtgatgtcaacttacacacatggtcagagggagactacttctggctcagttctctcagctgtctttctggtcctcggtcattcttttattcatctggtgctagggatgactcccaaggcttctgctgtgaggttaatattctgctgttgaactgtatattctaaatccttccataccatgtggaaggtaaatagaaaatatcatttctctgcgatctgctttttttttttttattaaacccagatggcagaagtggttcctggccttaccactatactacttcattggtatattggtccagttcacttctgaattatctttttaaggaggtgatttcagctctgatagatgaagccactgaggaagagtccagatggtgtgctcatggtcgttttagaggacaagggggtgaagatggggttagacgggaggccttttgtttaagggttcatgtatttcttaccaaaatggtaacaaaagatgagcagaaggggagggggatagagttatgcctatagtttctccttctgaagatgtaagttccagaaatatggatcatggacatatagttagacactacagaaccacacatttcatcttccagtgaagatggtagttacagtggggcatacccataaatccatcactcaagaccagagagaggcaggaggtctgaactttaagatcaaccatattatctattgtatagatagtaaagtcaagcctatcttgggttaaaaaattgttggtgcatttaagagcagtgtccacagcggccttcccctggaagtggagttacagggcttcgagtggctggatgtgggtgggtgctctccagggaggaagctagccactgcgcagacaagcccacttttgtttgttttgttttgagactggctttctctgtgtggctctggctgtcctagaactccactgtatagacttggctgacctcaaactgagatctgcctgcctctgcctccttagtgtaagggactgcaggcatgcgccaccaccgcctacctttttcctttttcaagaagattttgtttttagaccaggctcaatacacagagatcctctggcttttgcattctgagtgctggaatcaaatgcatgcatgccacagcctctgccagactcaaaagatttttttttttttaaagttgtgtttgtgcatgtgagtgcaatgcccatagaggccagaagaggctgtaggaccccctggaagtggaattacaggggcttggagcggctggatgcgggtgctggcaagcaaaccagagtcatcctctgcaagagcagcactacctgctctcaagtgccgagccacctcttcaggcaagggggaggtgggtggcgccagtcgcagcacggacgagcccactatagccaggaggtgtgggtgcctccgttgcgcgcatgcgcactcccttcgggcatagcatggtcttcccccactctcttgccctgtgtggtacatgctgcttccgcttgcgccgccccctcccaattggtttccgcgcgcgcgaaaaagccggggtctcgttcagagctgttctgtcgtctgcaacctgcaagatgccagcgcgaacagctccagcccgagtgcctgcgcttgcctccccggcaggctcgctcccggaccatgtccgcaggcggtaggtgccacgcagggtgggggtgaggggcgggaccgatgccgaggcatatattgggggtatctccgggtggccctgcggccgctgacagccagggcgcgcatgcgcggggcagcgtttggcgcgggctgtgcttcctgtcgcgcgtgcccttagctgttggcgccaaaatgggtgggggccggggtgctggcaagcccattgactgggagcttgagggagcaaggggatcagccctctgaaatagaacaagcttggggacaccctcaggggttcccctctttggtcagccaggctcctgggtgtgcaggaagtgcccctctcccggcctgtggacaccctgaagcccccactccgctccagcctccacttccggtgccctgcagatcccgccgcgcccacccgagggtccctccgccggttcccctggccctggttcagaagggccctccttcccagccccgctgtcaagtcctaggaccttttctctctcatcctgccgggccgggcctgggcagcttgaagccctcgtgggattttgttttgttttcatcctaacgtaaaggagctcataacttcgtataatgtatgctatacgaagttatgcccgtggccactagtacttctcgagctcgaattcgtcgacctcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatcttggtggcgtgaaactcccgcacctcttcggcagcgccttgtaaagcgcgtatggcttcgtacccctgccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacggatcggggtcgatgaatcagatccccgggatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctggtcgactctagatgcggccgcatgcatcctaggcctattaatattccggagtatacgtagccggctaacgttaacaaccggtggcgcgccataacttcgtataatgtatgctatacgaagttatgctcctgcgatcctggagctatcaaagagactctgcctggaaagagctggtgctgcagaatgggccctccactttggcagtgacataactctgtcccgagatcccagagcaatcgaggaaacctttaactccattcaaagtgtttatcagggggaaggaaagatgaatggtagtgagatgtctcggcccccgccctattattttagcccctgtaaaccagttctttcggtcctggcgccatcgaggaagctttttacattgtccagtgtttatttcgaggagtgagggatggcaggtaaagtcccccatttcagtgcatatgaatcccccctctcccccccccctccccaagcaatcaaggaagacctgggtaatgggccctccactagtaccttccacgcctccatgaggacggctgtgtgcaaggattggcgggaacactggatcccgcttcctgttccagaagggcatcgtcaatccgttcattcctgaaagcttgtcccgcgcgtcattgagcattagtcataggtgccctgctagtgctggacacatcccaacccgattctgagtctgcttttgctatcccgggggttagtttactggggtgatgccaggccgattcccatttgagctttgggggataaagtgggagaagcggctgaattccccttgtcaaacacccctgcccccagctcgcccccagggttggacctgaagctctgtactgagctacaccttcagccctttcacttgtattggagactggcctgggattctctctctctctctctgcctgtgtcttctcagtgctgggattactgacatgtgccatcatgatggtttgggttcgttttttgaatgcttgttttttgattgtataggcataaaaatctgaagcctttgtgagacacctcaacagatttctttttactaccaaaaaatacatacataattaaagtatttggttttttaaagttagacagtctgtaacctgcctttctgtcttttgtctttcctgttctcgggttggttggtttacctgtgtatgctttgttttcatttccaggaagggattcctgtaggccaagctagcctccggctggccgtctaacccaggctagccttggtcttttgtatctacccaagtgctgggattacaggaatgtgacactgtgcctagcttagcttttcttgatttagaaaaacttactagcaaaatatctacagtatgagatttgagaggtacctgcatttgggaggatagtccaggagctgagtaggcctcgaggtagcttccgtccatgctgctattctgtatttaaatggcagtttgtttaaatagaagtgtgcatagttcctctggatatataaaatatagtatcttcaacatttggattgtaagtctttaagtgtgtatgtgggtctatgctacagcttgtgtggcagtcaggaccccttgcaggaactggcctttccacaagattctgggtatgggaactcagaccatcaagcttggtagcattcctcttatctcaccagccccagactgacagtttctctgaaatatagtaaaccttgtgtcattcaaaaatcaaatcatgctttttagtgagatgttgtggccatggcctgtaacatcaggaggcaggagcaggagggtcacaagttagaattcaactaaggtctcatttcagacattcacaccttgcaaggtgagggatttcttttcctgagcagggggtgtcctttccatagctgagattccttggttggcatgggtttttcatttctttcagtttttttaaattaaaacttgttttccaggcacggtttctgtgtagttcatctggtcttgatcttgccacgctcgtgcctctggagtgatgcgtgttacatgcttggcttgcttttaacttattttttgttaaacctgtgtataagtggtgtgtgtgatatacatatatacacatatatatgtacatatacacgaaggtatgtgttaggggacagctttttaattttctgtgcatatgcaccacagtggcatatagaggtcttcgtggcagttaggggtctttgtggtgtttattactctctcacctctatgtggatttgggatcaaactgaggcttgtcaagaatgtgtaacaagtgcccgtctcaccaagccatgggtaactacgtttatctgtttgtctgtacagtgtatggatggagttggaaaacgcctttcttgagtcagttcttaggggtcctggagatggatctcaggtcgtgggacgtgacaacctgtgtctttgtctgtggagccatttcatctgaccgtaaacttttatttcattttgagacagggtcttaccatgtagcctcagctaacttggaactctgtagaccagattgcccttgaactcaaatatccacctgcctttgcctcttgaatactgggcttagagacatgtgctattatggccagcttaaaaagctttatctatctatctacctatctatctatctattttagaattacatttgaactttttggaattggtttcttgccttctaccttatgctgagaggggggggggtggtggaggaggcaggttgattacttgtgtaggtgctttgctgtgagtctctgtgcaccacgtgtgtgcctggtgctggagccctacaaatggattttcactttcaaccttacttgctttttggattttgtttgtttgagacaaggtttcactatagaaccccagctgttgtagaacttggcctggagctaacaaacccttctcctacctgtgcctctgagtaggggactgataacctttgctttgggaccatttgcttataaccttgcagcacttgattcagttagtctgcctggccagcaggctcaggggacctttctcaggtccatgcctgtctttttgagttttgagatccaaactcctgtccttgtgcttgtgtgacagtcacttcactgacctccgaagcccttttctgatggagttttccagacagttgggtagctggggctggcctcccatttcctgtgtagcttaagacggcatgaactcctcatcctcctgcctccactacaggcgctggcgtccgtggcttctgtggagcctcagctgcgctaagcaagcgctccaggtgctgattttctccctctctgtatttaggatcacgctgcagaaggctgatacagcatggcttaactcattaggaggcgggccgagttctaggacttaaactttatttttgctgtgctgaagttgatctcaggcctgtatatctgttagctctctggtggagggggtggggagtgggggtacctctctctgcagcagatcctatgctgtcccttacagtggtgtagttgtcattttttgtttgtttgttttcgagacagggtttctctgtatagccctggctgtcctggaactcactttgtagaccaggctggcctcgaactcagaaatccccctgcctctgcctcccgagtgctgggattaaaggcctgcgccaccacgcccggctgcagttgtcatcttaatcttggtgccctgtccatgtccatttattatctgtgtgtgtcatgtgcatgcatgtgcccattagagaccacaggaaggggttgggtccctcggagctggagttacatagggagagggtgcttgctgtaagctgcctgacccaggtgcagagcaatgcggccacgaagcagtctctctagggctccccatccattctgggcgggggtggggggttactggggttagaactcagggcagctattttaccactcagtgagctatctgaaaaatggggctggggctgactgctcctcagggttctgtaggcctgctgaactcgtggtagtaggttaggtctccaccctgggccatcgcagcagtgagcagggagtgtctacaatatattcaggtacgctgtgcagtagactgttggattttattccaagctcgcttgtagaatgatctaggcttgttctttctgcagtttgcctaggcggtttgtgtgccagatctcaagtcacccttctgtttttaaactgggtctttgtagcccagtccatttaactctctgacctgctgcctcagccttctgagtgctgctggggttgccggcatgagccgccaagcctggtttagcatgtagctcttttctcagaggttgaggtgactttctgtctatcttctcagtgtggcagtggagctggggctctcagacatgccaggcaggtgctgtgcctctgagccacaccttgggacagacacttgctgttctcttgctctcgttctgtaggtcaggaattgaggggtaggatgggtagagaagaaacctagatgtcctgggctggattccttctggaaacgggcagggagtctgtcctcctggggagctgacttctagtttcttgctgtttgtgggaaatgtttttgtttttcatggcagggtttatctctgtagccctggctgagaatgtagatcaggctggccttgaactcggatcacctgcctgcttgccactgcctcccaagtgctggaaacaaggcatgtgccaccgtgcttggccctaatttcttttttgtaatttcaaggcagggtctagttgtatacttccaggtgatcttgaactcggcttccctcctgcagccttgtgagcctgtgccaggtgtgtattacatctgcatacgtagtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 12 - DNA, 1122 bp, Artificial Sequence
  modified lacI with the stabilizing amino acid and the tight binding mutation
atgggcaaatatgtaacattgtatgatgttgcagagtatgccggtgtctcttatcagactgtttccagagtggtgaaccaggccagccatgtttctgccaaaaccagggaaaaagtggaagcagccatggcagagctgaattacattcccaacagagtggcacaacaactggcaggcaaacagagcttgctgattggagttgccacctccagtctggccctgcatgcaccatctcaaattgtggcagccattaaatctagagctgatcaactgggagcctctgtggtggtgtcaatggtagaaagaagtggagttgaagcctgtaaagctgcagtgcacaatcttctggcacaaagagtcagtgggctgatcattaactatccactggatgaccaggatgccattgctgtggaagctgcctgcactaatgttccagcactctttcttgatgtctctgaccagacacccatcaacagtattattttctcccatgaagatggtacaagactgggtgtggagcatctggttgcattgggacaccagcaaattgcactgcttgcgggcccactcagttctgtctcagcaaggctgagactggccggctggcataaatatctcactaggaatcaaattcagccaatagctgaaagagaaggggactggagtgccatgtctgggtttcaacaaaccatgcaaatgctgaatgagggcattgttcccactgcaatgctggttgccaatgatcagatggcactgggtgcaatgagagccattactgagtctgggctgagagttggtgcagatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgccagtctcactggtgaagagaaaaaccaccctggcacccaatacacaaactgcctctccccgggcattggctgattcactcatgcagctagcaagacaggtttccagactggaaagtgggcagagcagcctgaggcctcctaagaagaagaggaaggtgtga